(19) United States
(12) Patent Application Publication
(10) Pub. No.: US 2024/0130674 A1
(43) Pub. Date: Apr. 25, 2024

(54) MEANS AND METHODS FOR SELECTING PATIENTS FOR IMPROVED PERCUTANEOUS CORONARY INTERVENTIONS

(71) Applicants: CV Cardiac Research Institute, Aalst (BE); Politecnico di Torino, Torino (IT)

(72) Inventors: Jeroen Sonck, Wemmel (BE); Carlos Collet, Brussels (BE); Maurizio Lodi Rizzini, Casalmaggiore (IT); Diego Gallo, San Damino d'Asti (IT); Umberto Morbiducci, Macerata (IT); Claudio Chiastra, Segrate (IT)

(73) Assignees: CV Cardiac Research Institute, Aalst (BE); Politecnico di Torino, Torino (IT)

(21) Appl. No.: 18/269,234

(22) PCT Filed: Dec. 23, 2021
(86) PCT No.: PCT/EP2021/087477

§ 371 Date: Jun. 21, 2023

(30) Foreign Application Priority Data
Dec. 23, 2020 (GB) 2020468.1

(51) Int. Cl. A61B 5/00; A61B 5/021; G06T 7/00; G06V 10/764; G06V 10/766; G16H 50/50

(52) U.S. Cl. CPC A61B 5/4842; A61B 5/021; G06T 7/0012; G06V 10/764; G06V 10/766; G16H 50/50; G06T 2207/10081; G06T 2207/10101; G06T 2207/10132; G06T 2207/30048; G06T 2207/30096; G06T 2207/30101

(57) ABSTRACT

The present invention provides a computer device and a computer-implemented method to quantify the extent of functional coronary artery disease. In addition, the invention provides a computer device for determining the functional pattern of coronary disease in a mammal. It is shown that a mismatch in the extent of CAD between anatomical and physiological evaluations is predictable for an improvement in epicardial conductance with percutaneous revascularization. More particularly the invention provides methods to select a mammal suffering from coronary disease to benefit from a percutaneous coronary intervention.

### FIELD OF THE INVENTION

The present invention relates to the field of cardiac disease, in particular to the assessment of coronary vessels, in particular to determine the patterns of blockage or restriction to the blood flow through a coronary vessel. More particularly, the present invention relates to a computer-implemented method to quantify the extent of functional coronary artery disease. In addition, the invention provides a computer device for determining the functional pattern of coronary disease in a mammal. More particularly the invention provides methods to select a mammal suffering from coronary disease to benefit from a percutaneous coronary intervention.

### INTRODUCTION TO THE INVENTION

Invasive functional assessment of coronary artery disease (CAD) has been regarded as the gatekeeper for revascularization in patients with chronic coronary syndromes. Guidelines advocate evaluating the reduction in coronary flow using pressure-derived indices to decide upon the need for revascularization. Intracoronary pressure measurements are typically performed in the distal segment of the coronary artery reflecting cumulative pressure losses along the epicardial vessel. Focal narrowing can be entirely responsible for the pressure drops; nonetheless, diffuse functional deterioration can be also observed outside angiographic stenotic regions contributing to the total decrease in coronary perfusion pressure. Coronary angiography remains to date the most utilized method to guide stent implantation. The length of the lesion can be quantified by quantitative coronary angiography (QCA) or alternatively, more precisely, using intravascular imaging. Both approaches aim to guide stent selection to cover the atherosclerotic plaque, restore epicardial conductance and improve myocardial perfusion. However, in almost a third of patients after an angiographically successful percutaneous coronary intervention (PCI), epicardial conductance remains suboptimal. In diffuse functional disease, PCI is of limited benefit in terms of coronary physiology whereas in focal CAD PCI usually restores epicardial conductance. Furthermore, patients with low fractional flow reserve (FFR) after percutaneous revascularization have been shown to be at an increased risk of adverse events compared to patients with high post-PCI FFR. Gain in epicardial conductance with PCI can be predicted by assessing the distribution of epicardial resistance. A pullback maneuver during intracoronary pressure measurements identifies the presence, location, magnitude and extent of pressure drops. Two factors, namely (i) the magnitude of FFR drops and (ii) extension of functional CAD are predictive of improvement in epicardial conductance after percutaneous revascularization. Thus, quantifying the extent of functional disease may have prognostic capability for post-PCI FFR.

In the present invention, we sought to quantify the mismatch in the extent of CAD between anatomical and functional evaluations and to assess the impact of functional-anatomical mismatch (FAM) on FFR after PCI.

### SUMMARY OF THE INVENTION

According to a first aspect, there is provided a computer device for quantifying the extent of functional coronary artery disease (CAD) comprising a processor configured to:
i) process a set of fractional flow reserve (FFR) values obtained at different positions of a coronary vessel between the ostium and the most distal part of the coronary vessel,
ii) classify the coronary vessel in healthy, focal and/or diffused diseased segments by carrying out a piece-wise linearization of said FFR data by applying an automated change-points detection algorithm.

This approach is that it is less vulnerable to artefacts in the pullback curves compared to prior art devices that do not make use of piece-wise linearization by means of an automated change point detection algorithm. It is clear that piece-wise linearization by means of an automated change point detection algorithm, by means of the parameters determined based on the linear segments is less sensitive to local artefacts, which are for example the result of temporary or local measurement errors, etc. Furthermore, when use is made of this improved quantification in the context of FAM, as explained in further detail below, which quantifies the mismatch between the anatomical and functional CAD length, and it also becomes possible to take into account the impact of residual pressure losses outside the treated region on post PCI physiology. Moreover, the FAM approach is based on the presence and length of disease rather than on the magnitude of pressure drops making this approach less influenced by the interaction in cases of serial lesions. Further this allows an improved assessment of the functional pattern of CAD which may improve patient selection for PCI by avoiding stenting lesions which don't result in sufficient post-PCI benefits, by reducing the risk of peri-procedural myocardial infarction and by increasing the chance of a net clinical benefit from revascularization. In this way patients with a negative FAM, i.e. having diffuse functional CAD, may be better treated with optimal medical therapy or coronary artery bypass grafting, and patients with a positive FAM may be better treated with PCI.

It is clear that said FFR data refers to said set of fractional flow reserve values.

According to an embodiment, there is provided a computer device, configured to:
ii) classify the coronary vessel in at least one of the following:
   one or more healthy segments;
   one or more focal diseased segments;
   one or more diffused diseased segments,
by carrying out said piece-wise linearization of said FFR data by applying said automated change-points detection algorithm.

According to an embodiment, there is provided a computer device, configured to:
ii) classify the coronary vessel in at least two of the following:
   one or more healthy segments;
   one or more focal diseased segments;
   one or more diffused diseased segments,
by carrying out said piece-wise linearization of said FFR data by applying said automated change-points detection algorithm.

According to an embodiment, there is provided a computer device, configured to:
ii) classify the coronary vessel in the following:
   one or more focal diseased segments; and
   one or more diffused diseased segments, and
   optionally, one or more healthy segments,
by carrying out said piece-wise linearization of said FFR data by applying said automated change-points detection algorithm.

According to an embodiment there is provided a computer device configured to quantify the extent of the functional coronary artery disease (CAD), or in other words the function lesion length, based on and/or such that it corresponds to:
the sum of length of the segments classified as diseased fragments;
the sum of the length of the focal diseased segments and the diffuse diseased segments;
the sum of the length of the segments characterized by FFR deterioration.

According to an embodiment the FFR data comprises the set of FFR values obtained at different positions of a coronary vessel between the ostium and the most distal part of the coronary vessel.

According to an embodiment the set of FFR values corresponds to an FFR pullback curve.

According to an embodiment there is provided a computer device is configured to quantify the functional lesion length from analysis of the FFR pullback curve.

According to an embodiment the computer device is further configured to perform the quantification of the functional lesion length and/or the classification of the coronary vessel in segments after one or more of the following:
smoothing the set of FFR values;
applying a moving average or mean filter to the set of FFR values;
applying a low pass filter to the set of FFR values.

According to an embodiment, there is provided a computer device, wherein the computer device further comprises a display configured to display said healthy, focal and/or diffused diseased fragments, optionally on an image of the coronary artery, optionally wherein the displayed image of the coronary artery is a 2-dimensional image.

According to an embodiment, there is provided a computer device, wherein the automated change-point detection algorithm is configured to detect one or more change points in the set of FFR values, such that said change points each correspond to a position along the coronary vessel where an attribute of the set of FFR values changes, wherein:
said one or more change points are configured to divide the set of FFR values in two or more segments, in which each change point defines an endpoint between two segments; and
said two or more segments, each corresponding to a linearized subset of the set of FFR values obtained at different positions along the coronary vessel between a proximal point of the segment and a distal point of the segment

According to an embodiment, there is provided a computer device, wherein:
said attribute is an average value and/or a slope; and or
said two or more segments are characterized by the following quantities:
   FFR drop, which is the difference between the FFR value at the distal point and the FFR value at the proximal point of the segment; and
   Segment length, which is the distance along the coronary vessel axis between the distal point of the segment and the proximal point of the segment, and
   Optionally segment slope, which is the ratio between the FFR drop and the segment length.

According to an embodiment, there is provided a computer device, wherein the computer device is further configured to classify the coronary vessel such that:
segments are classified as healthy segments or as diseased segments by means of a predetermined first classification threshold function based on the FFR drop, the segment length and/or the segment slope of the segments; and
optionally, diseased segments are classified as:
   focal diseased segments or as diffuse diseased segments by means of a predetermined second classification threshold function based on the FFR drop, segment length and/or segment slope of the segments; and
optionally, segments as classified as healthy when said segments exhibit a positive FFR drop and when said segments are contiguous to a diseased segment and said segments are shorter than 30 mm, preferably 25 mm, and even more preferably 20 mm; and
optionally the computer device further comprises a logistic regression model configured to automatically discriminate each segment as a healthy segment, a focal diseased segment and/or a diffuse diseased segment, optionally a two-variables logistic regression based on the FFR drop, the segment length and/or the slope of the segment, optionally, wherein the logistic regression model is determined from visual adjudication of a derivation cohort, configured to discriminate between healthy and diseased segments, and further to discriminate between focal diseased segments and diffuse diseased segments.

According to an embodiment, the logistic regression model is configured to provide a binary separation.

According to an embodiment, the computer device is further configured to apply the logistic regression model in a two-steps approach, in which:
in step 1 the logistic regression model is configured to classify, by means of separation of the segments into healthy segments and diseased segments, wherein the diseased segments comprise the focal diseased segments and the diffuse diseased segments; and
in step 2 the logistic regression model is configured to classify, by means of separation of the diseased segments into focal diseased segments and diffuse diseased segments,
thereby providing an automatic adjudication of the segments of the piece-wise linearized FFR data, preferably an FFR pullback curve.

According to an embodiment, there is provided a computer device, wherein said automated change-points detection algorithm is configured to operate based on a penalized parametric global method.

According to an embodiment, there is provided a computer device, wherein the display is further configured to display the image of the coronary artery in a 2-dimensional image.

According to an embodiment, there is provided a computer device, further configured to obtain the set of FFR values from:
a pull-back curve; or
a 3-dimensional quantitative coronary angiography; or
a CT scan; or
intravascular imaging, optionally an optical coherence tomography (OCT) or an intravascular ultrasound (IVUS); or
the combination between coronary angiography and intravascular imaging; or
the combination of a CT scan and intravascular imaging; or
computational fluid dynamics simulations applied to a 3D model of the coronary vessel as reconstructed from medical imaging, optionally wherein the medical imaging comprises: a 3-dimensional quantitative coronary angiography, a CT scan, an OCT or an IVUS.

According to an embodiment, there is provided a computer device, wherein the computer device is further configured to predict the response to a percutaneous coronary intervention (PCI) by said quantifying of the extent of functional CAD, and/or wherein the computer device is further configured to quantify the extent of functional CAD as the sum of the lengths of the diseased segments.

According to an embodiment, there is provided a computer device, wherein the computer device is further configured to select a mammal suffering from coronary artery disease (CAD) to be eligible for a percutaneous coronary intervention (PCI) by said quantifying of the extent of functional CAD, and selecting a mammal when the extent of functional disease in the coronary artery is smaller than the extent of anatomical disease in the coronary artery; and/or wherein the computer device is further configured to calculate a Functional Anatomical Mismatch (FAM) as the difference between the extent of anatomical CAD and the extent of functional, thereby identifying two lesion endotypes: (1) functional CAD circumscribed within the anatomical CAD when FAM>0, and (2) functional CAD extending beyond the anatomical CAD when FAM<0.

According to an embodiment, there is provided a computer device, wherein the computer device (system) is configured to operate offline.

According to an embodiment, there is provided a computer device, wherein the computer device is configured to perform said automatic classification.

According to a second aspect, there is provided a computer-implemented method to quantify the extent of functional coronary artery disease (CAD) comprising the following steps: i) processing a set of fractional flow reserve (FFR) values obtained at different positions of a coronary vessel between the ostium and the most distal part of the coronary vessel, ii) classifying the coronary vessel in healthy segments, focal diseased segments and/or diffused diseased segments by carrying out a piece-wise linearization of said FFR data by applying an automated change-points detection algorithm, and optionally iii) displaying said healthy, focal and/or diffused diseased fragments on an image of the coronary artery, and optionally said automated change-points detection algorithm is based on a penalized parametric global method.

According to an embodiment, there is provided a computer-implemented method, wherein said method comprises the step of obtaining the set of FFR values from a pull-back curve, or 3-dimensional quantitative coronary angiography, or a CT scan, or intravascular imaging (e.g. optical coherence tomography (OCT) or intravascular ultrasound (IVUS), or the combination between coronary angiography and intravascular imaging or the combination of a CT scan and intravascular imaging.

According to a third aspect, there is provided a computer-implemented method for developing an automated classifier for use in the computer device according to the first aspect for performing the classification of the coronary vessel in healthy focal and/or diffused diseased segments and/or for use in the computer-implemented method according to the second aspect for performing the classification of the coronary vessel in healthy focal and/or diffused diseased segments, wherein:
the automatic classifier is developed based on logistic regression, preferably two-variables logistic regression based on the FFR drop, the segment length and/or the slope of the associated segment; and
optionally the logistic regression is determined from visual adjudication of a derivation cohort, configured to discriminate between healthy and diseased segments, and further to discriminate between focal diseased segments and diffuse diseased segments.

The mismatch between anatomy and physiology regarding epicardial lesion severity has been widely recognized in the prior art. For example, in the FAME study, more than one-third of lesions with an angiographic 50% to 70% diameter stenosis demonstrated an FFR 50.80 whereas one-fifth of lesions with a 71% to 90% angiographic diameter stenosis demonstrated an FFR>0.80. Disconnection between anatomy and physiology goes beyond the assessment of lesion significance. The length of CAD also differs between anatomical and functional evaluations. In the present invention, we have determined the length of functional CAD lesion with the means of a specially developed automatic algorithm. Accordingly, our novel computer-implemented method shows that when the length of functional disease (in a coronary artery of a patient) is greater than its anatomical equivalent either derived from QCA or optical coherence tomography (OCT) then the FAM value is <0 (see FIG. 6). Accordingly, when the FAM value<0 there is no beneficial effect for carrying out a PCI.

In one aspect the invention relates to a computer-implemented method to quantify the extent of functional coronary artery disease (CAD) comprising the following steps: i) processing a set of fractional flow reserve (FFR) values obtained at different positions of the coronary vessel between the ostium and the most distal part of the coronary vessel, ii) classifying the coronary vessel in healthy, focal and/or diffused diseased segments by carrying out a piece-wise linearization of said FFR data by applying an automated change-points detection algorithm and optionally iii) displaying said healthy, focal and/or diffused diseased fragments on a 2-dimensional image of the coronary artery.

In specific aspects in the computer-implemented method the FFR values are obtained from a pull-back curve, 3-dimensional quantitative coronary angiography, CT scan or OCT. It is thus clear that the set of FFR values, or in other words the FFR data or FFR pullback curve, according to an embodiment can be obtained as data that was measured, generated and/or recorded from measurements of suitable pressure sensors during an FFR pullback operation, or in other words FFR data obtained from pressure measurements in the coronary artery vessel, which is an invasive measurement. It is however clear that preferably the embodiment of the computer implemented method does not include the invasive step of making the pressure measurements in the coronary artery vessel, and preferably only processes data received as an input, resulting from such measurements. According to alternative embodiments, the set of FFR values, or in other words the FFR data or FFR pullback curve does not result from direct pressure measurements inside the coronary vessel but is calculated by means of computational fluid dynamics simulations applied to a 3D model of the coronary vessel as reconstructed from medical imaging, such as for example 3-dimensional quantitative coronary angiography, CT scan, OCT or IVUS. It is clear that according to such an embodiment, the FFR data can be obtained by means of non-invasive measurements, such as for example 3-dimensional quantitative coronary angiography, CT scan. When according to such embodiments, there is made use of invasive measurements, such as for example OCT or IVUS, it is clear that preferably the embodiment of the computer implemented method does not include the invasive step of making the measurements in the coronary artery vessel, and preferably only processes data received as an input, resulting from such measurements, and preferably the medical imaging data from these measurements, or a 3 dimensional model of the coronary vessel as reconstructed from such medical imaging data.

In another aspect an in vitro method is provided to predict the response to a percutaneous coronary intervention (PCI) by quantifying the extent of functional CAD.

In yet another aspect an in vitro method is provided to select a mammal suffering from coronary artery disease (CAD) to be eligible for a percutaneous coronary intervention (PCI) comprising the application of the computer-implemented method described herein and selecting a mammal when the extent of functional disease in the coronary artery is smaller than the extent of anatomical disease in the coronary artery.

In a specific aspect the method is an offline method.

In yet another aspect the method to quantify the extent of functional coronary artery disease is an automatic classification method.

In another aspect a computer device is provided for evaluating the functional pattern of coronary artery disease in a mammal, said computer device configured to process a set of FFR values obtained at different positions of the coronary vessel between the ostium and the most distal part of the coronary vessel and classifying the coronary vessel in focal and/or diffused diseased segments by carrying out a piece-wise linearization of said FFR data by applying an automated change-points detection algorithm.

According to a further aspect, there is provided a computer-implemented method to quantify the extent of functional coronary artery disease (CAD) comprising the following steps: i) processing a set of fractional flow reserve (FFR) values obtained at different positions of the coronary vessel between the ostium and the most distal part of the coronary vessel, ii) classifying the coronary vessel in healthy, focal and/or diffused diseased segments by carrying out a piece-wise linearization of said FFR data by applying an automated change-points detection algorithm and optionally iii) displaying said healthy, focal and/or diffused diseased fragments on an image of the coronary artery.

According to an embodiment, there is provided a computer-implemented method wherein said automated change-points detection algorithm is based on a penalized parametric global method.

According to an embodiment, there is provided a computer-implemented method wherein in step iii) the displayed image of the coronary artery is a 2-dimensional image.

According to an embodiment, there is provided a computer-implemented method wherein the FFR values are obtained from a pull-back curve or 3-dimensional quantitative coronary angiography or CT scan or intravascular imaging (e.g. optical coherence tomography (OCT) or intravascular ultrasound (IVUS) or the combination between coronary angiography and intravascular imaging or the combination of a CT scan and intravascular imaging.

According to a further aspect, there is provided a method to predict the response to a percutaneous coronary intervention (PCI) by quantifying the extent of functional CAD according to the previous aspect.

According to a further aspect, there is provided a method to select a mammal suffering from coronary artery disease (CAD) to be eligible for a percutaneous coronary intervention (PCI) comprising the application of the computer-implemented method according to a previous aspect and selecting a mammal when the extent of functional disease in the coronary artery is smaller than the extent of anatomical disease in the coronary artery.

According to an embodiment, there is provided a method according to a previous aspect wherein the method is an offline method.

According to an embodiment, there is provided a method according to a previous aspect wherein the method is an automatic classification method.

According to a further aspect, there is provided a computer device for evaluating the functional pattern of coronary artery disease in a mammal, said computer device configured to process a set of FFR values obtained at different positions of the coronary vessel between the ostium and the most distal part of the coronary vessel and classifying the coronary vessel in healthy, focal and/or diffused diseased segments by carrying out a piece-wise linearization of said FFR data by applying an automated change-points detection algorithm, optionally said automated change-points detection algorithm is based on a penalized parametric global method.

According to an embodiment, there is provided a computer device wherein the set of FFR values are obtained from a pull-back curve, or 3-dimensional quantitative coronary angiography, or a CT scan, or intravascular imaging (e.g. optical coherence tomography (OCT) or intravascular ultrasound (IVUS), or the combination between coronary angiography and intravascular imaging or the combination of a CT scan and intravascular imaging.

### FIGURE LEGENDS

Exemplary embodiments will now be described, for example with reference to the following Figures. When in the context of these Figures, and/or the description, there is made reference to colors as present in the drawings as appended to the filing of this patent application. These colors have been translated into corresponding indications by means of different line styles in the following Figures.

FIG. 1: Definition of QCA-derived anatomical lesion length, functional lesion length and functional-anatomical mismatch (FAM_{QCA}). A): The QCA-derived anatomical lesion length was obtained starting from two angiographic projections for each target lesion. B): 3D quantitative coronary angiography algorithm was used to obtain the QCA-derived anatomical lesion length as the distance where the reference diameter line intersects with the curve describing the local vessel diameter value. In other words, as shown in FIG. 1, a set of values representing the diameter of the coronary vessel or the lumen of the coronary vessel is determined at different positions between the ostium and most distal part of the coronary vessel, which could also be referred to as the proximal and distal position of the coronary vessel. Preferably the values for the diameter of the coronary vessel are determined automatically, by means of the processing of the image data of the coronary angiography by means of quantitative coronary angiographic (QCA) algorithms, which are for example configured to automatically detect the contour of the vessel and derive the diameter therefrom. It is clear that alternative embodiments are possible for determining the anatomical lesion length, in which the diameter, or any other suitable parameter indicative thereof, such as for example lumen area, etc. is determined along the length of the coronary vessel. As further shown, in order to determine there is determined a reference dataset for the diameter of the coronary vessel, which according to this embodiment is represented as a linear function, which corresponds to a normal reference for the evolution of the diameter of the vessel along the length of the coronary vessel. According to some embodiments, the reference data set could comprise an interpolated set of reference data derived from the data representing the diameter of the coronary vessel along the length of the coronary vessel. It is clear that alternative embodiments are possible, such as for example a predetermined set of reference data determined in function of the dataset with the actually measured diameter of the vessel and/or any other suitable parameters such as for example a specification of the coronary vessel, patient characteristics, etc. As further shown in the embodiment of FIG. 1B the QCA defined anatomical length is determined by the length of sections which experience a reduction of the actual diameter with respect to the reference diameter. In other words, according to the representation shown in FIG. 1B, the length between two points where the reference diameter intersects the line representing the values of the actual diameter, and between which the line representing the values of the actual diameter remains below the line of the reference diameter. It is clear that, when a plurality of such portions of the line of the actual diameter remaining below the reference line between two respective intersection points are available, according to an embodiment the anatomical length can be determined as the aggregation of the respective lengths of the plurality of such portions. According to a preferred embodiment, such as shown in FIG. 1B, only the length of such portions is taken into account for aggregating the anatomical length when, according to suitable parameters the portion qualifies as an anatomical lesion. According to one embodiment, such a portion is qualified as a lesion, for example based on the minimal diameter of such a portion, the maximum diversion from the reference diameter of such a portion, the length of such a portion, or any other suitable parameter or combination of parameters. As shown in FIG. 1B, the QCA defined anatomical length is determined by means of the portion that is indicated by means of the box, in other words the length between the two intersection points of the line of the reference diameter with the line of the actual diameter for the portion of the reference diameter that stays below the line of the reference diameter. As shown further portions of the curve of the actual diameter which are below the reference diameter line are not taken into account for the calculation of the QCA defined anatomical length, for example based on the minimal lumen diameter of these portions. Such a detection and calculation of the QCA defined anatomical lesion length can be performed by means of a suitable automated computer-implemented method, for example based on machine learning techniques for automatic identification of lesions on coronary CT images. According to an embodiment, such computer-implemented methods may make use of support vector machines, or any other suitable method, which are operating on data relating to quantitative geometric and shape features of the coronary artery vessel, such as for example the lumen diameter of the coronary artery vessel, minimum lumen diameter, and/or any other suitable parameter, such as for example circularity, eccentricity, . . . to automatically detect and/or quantify portions of the coronary vessel that qualify as anatomical lesions. It is clear that alternative embodiments are possible, in which for example use is made of suitable thresholds, deep learning, etc. It is however clear that the anatomical length of the CAD, could be determined from any suitable conventional angiography and corresponds to the length or extent of a stenotic segment of the vessel, or in other words the length of the CAD as identified by means of a predetermined reduction in the diameter or lumen area of the vessel. According to the embodiment shown in FIG. 1B the QCA-derived anatomical lesion length was automatically calculated using the 3D QCA software. As schematically shown by means of the box in FIG. 1, the anatomical lesion length is defined as the length between two points where the reference diameter line intersects the line representing the values of the actual diameter of the vessel along its length. According to the embodiments, shown, this calculation is performed automatically. According to this preferred embodiment, as shown by means of the box in FIG. 1B, preferably, as explained above only the length between two intersections is taken into account as determined by means of a suitable automatic computer-implemented method, for example the portion comprising the minimal lumen diameter of the vessel. When serial lesions are present in the vessel, it is clear that according to particular embodiments the anatomical length could be defined as the aggregation of the length of the two or more sections of the vessel between such intersections of the diameter values with the reference line. Similar as above, also for serial lesions, the sections that qualify as lesions could for example be determined by means of a suitable automatic computer-implemented method, for example based on the minimal lumen diameter of these sections. According to a particular embodiment, there could be automatically detected the presence of serial lesions, when there is detected the presence of at least two stenosis along the vessel, for example in which the quantitative parameters of the portion qualify as a lesion as determined by means of a suitable automatic computer-implemented method, for example based on the minimum value of the diameter of that portion, and which are positioned at a distance from each other of at least three times the reference vessel diameter. It is however clear that according to alternative embodiments other suitable automated computer-implemented methods for determining whether such a section qualifies for determining the anatomical lesion length are possible. C): The functional lesion length was obtained from analysis of the FFR pullback curve after smoothing and piece-wise linearization as the sum of the segments characterized by FFR deterioration. In other words, as described further below, the functional lesion length, or the length or the extent of the functional coronary artery disease (CAD), corresponds to the sum of length of the segments classified as diseased fragments characterized by FFR deterioration. In other words, as described further below, the functional lesion length, or the extent of the functional CAD, corresponds to the sum of the length of the focal diseased segments and the diffuse diseased segments. As will be explained in further detail below, the FFR deterioration is determined by means of characteristics of the respective segments such as for example FFR drop, segment length, slope, etc. as explained in further detail below. D): The FAM_{QCA} is defined as the difference between the QCA-derived anatomical lesion length minus the functional lesion length. It is clear that the functional lesion length, could also be referred to as the length or extent of the functional coronary artery disease. It is clear that according to alternative embodiments, the anatomical lesion length, or the extent of the anatomical coronary artery disease, could be determined by alternative means then the QCA, such as for example a CT scan, an optical coherence tomography (OCT), etc. as will for example be described in further detail below.

FIG. 2: Positive vs. negative FAM_{QCA}. A): example of vessel with positive FAM_{QCA}, where the QCA-derived anatomical lesion length is longer than the functional lesion length, respectively blue and red shade in the area and FFR curves (left panel). From left to right, FFR is displayed as a color-coded map on the 3-dimensional geometric reconstruction of the vessel. The color-coded map of FIG. 2 comprises the following sequence of colors as represented by line styles from top to bottom, for FFR: blue from 1.00 going into green from 0.8 to 0.6 going into red to 0.4; and for FAM_{QCA}: red from 20 mm going into yellow and green about −50 mm going further into to blue up to −120 mm. The extension of the anatomical and functional length is displayed in black, with indication of the relative FFR drop within the QCA-derived anatomical lesion. FAM_{QCA} is displayed as a color-coded map: the red color underlines that the functional disease was circumscribed within the anatomical lesion. The percutaneous coronary intervention (PCI) restored epicardial conductance and resulted in high post-PCI FFR (right panel), with a relative gain equal to 0.99. B): example of vessel with negative FAM_{QCA}, where the anatomical lesion length is shorter than the functional lesion length, respectively blue and red shade in the area and FFR curves (left panel). From left to right, FFR is displayed as a color-coded map on the 3-dimensional geometric reconstruction of the vessel. The extension of the QCA-derived anatomical and functional length is displayed in black, with indication of the relative FFR drop within the anatomical lesion. FAM_{QCA} is displayed as a color-coded map: the blue color underlines that the functional disease extended beyond the anatomical lesion. The percutaneous coronary intervention (PCI) resulted in minor improvement of epicardial conductance and a low post-PCI FFR (right panel), with a relative gain equal to 0.22.

FIG. 3: Definition of QCA-derived anatomical length, OCT-derived anatomical length, FAM_{QCA}, and FAM_{OCT}. An example of vessel with functional diffuse disease is considered. FAM_{QCA} is defined as the difference between QCA-derived anatomical length (panel A) and functional length (panel B) while FAM_{OCT} is defined as the difference between OCT-derived anatomical length (panel C) and functional length (panel B). The anatomical lesion derived from QCA or OCT is represented on the 3D geometric vessel reconstruction and color-coded using FAM_{QCA} or FAM_{OCT} (panels D and E, respectively). In both cases, functional lesion length is longer than anatomical lesion length (i.e. negative FAM).

FIG. 4: Development and performance of the automatic classifier. Healthy, focal disease and diffuse disease segments (green, red and blue, respectively) in length vs. FFR drop plane. The visual adjudication by two independent observers (CaC and SN) in the derivation set was used to develop the automatic classifier able to discriminate among healthy, focal disease and diffuse disease segments (panel A). The automatic classifier was then applied to the validation set (panel B). The performance of the classifier was evaluated by comparing with the visual adjudication by the two independent observers.

FIG. 5: Scatter plots illustrating the correlations among functional length and QCA-derived anatomical length, OCT-derived anatomical length and FFR relative gain. The QCA-derived anatomical length was not correlated with functional length (panel A). The OCT-derived anatomical length was correlated with the functional length (panel B). The functional disease length was inversely correlated with the FFR relative gain (panel C).

FIG. 6: Scatter plots illustrating the correlations among FAM, FFR relative gain and among the FFR drop within the anatomical lesion and FFR relative gain. A direct significant association was found between the FAM_{QCA} and the FFR relative gain after PCI, i.e. the larger the FAM the higher the functional relative gain after PCI (panel A). A direct significant association was also found between the FAM_{OCT} and the FFR relative gain after PCI (panel B). The FFR drop within the QCA-derived anatomical lesion was strongly correlated with the functional relative gain after PCI, i.e. the larger the drop is attributable to the anatomical lesion with respect to the functional lesion, the better the PCI outcome (panel C). The FFR drop within the OCT-derived anatomical lesion was strongly correlated with the functional relative gain after PCI (panel D).

FIG. 7: Explanatory case of FFR pullback curve with the generic points (m, FFRₘ), (i, FFRᵢ) and (n, FFRₙ).

FIG. 8: Explanatory case of piece-wise linearized FFR pullback curve with a graphical explanation of FFR drop and segment length.

FIG. 9: Results on the left are relative to observer CaC, on the right to observer SN. Panel A: Healthy segments, focal disease segments and diffuse disease segments (respectively, black, white, and gray; originally respectively, green, red, and blue) in length vs. FFR drop plane for the validation set. Panel B: confusion matrices of the classification healthy vs pathological segments. Panel C: confusion matrices of the classification focal vs diffuse disease segments.

FIG. 10: Sensitivity analysis to serial lesions of FAM_{QCA} and FAM_{OCT}. Considering only the contiguous segments for the definition of the functional length, a direct significant association was found between FAM_{QCA} and FFR relative gain after PCI (panel A), and between FAM_{QCA} and FFR relative gain after PCI (panel C). Excluding serial lesions from analysis, a direct significant association was found between FAM_{QCA} and FFR relative gain after PCI (panel B), and between FAM_{QCA} and FFR relative gain after PCI (panel D).

### DETAILED DESCRIPTION OF THE INVENTION

The present invention will be described with respect to particular embodiments and with reference to certain drawings, but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention. Unless defined otherwise, all technical and scientific terms used herein have the same meaning as commonly understood by one of ordinary skill in the art (e.g. in molecular biology, interventional cardiology fluid physics, biochemistry, and/or computational biology/biomechanics).

Functional complete revascularization of the coronary arteries has been associated with improved clinical outcomes after percutaneous coronary interventions (PCI). Nevertheless, in one third of patients, coronary perfusion pressure remains low even after a successful procedure. In the present invention we developed a computer-implemented method which was able to quantify the mismatch in the extent of CAD between anatomical and functional invasive evaluations. In the invention we have assessed the impact of this mismatch on post-PCI fractional flow reserve (FFR).

Accordingly, the invention provides in a first embodiment a computer-implemented method to quantify the extent of functional coronary artery disease (CAD) comprising the following steps: i) processing a set of fractional flow reserve (FFR) values obtained at different positions of the coronary vessel between the ostium and the most distal part of the coronary vessel, ii) classifying the coronary vessel in focal and/or diffused diseased segments by carrying out a piece-wise linearization of said FFR data by applying an automated change-points detection algorithm based on a penalized parametric global method and optionally, and iii) displaying said focal and/or diffused diseased fragments on a 2-dimensional image of the coronary artery.

In a particular embodiment the FFR values are obtained from a pull-back curve, 3-dimensional quantitative coronary angiography, CT scan or optical coherence tomography (OCT).

Automatic change-point detection algorithms are known in the art but have never been used in the context of cardiac disease. Several of such algorithms are known in the art. For example, the binary segmentation method proposed by Scott, A. J. and Knott, M. (1974) Biometrics, 30 (3):507-512 is a well-known changepoint search method. Yet another approach is based on the algorithm of Jackson B. et al (2005) IEEE, Signal Processing Letters, 12(2):105-108. In the present invention the method of Killick R et al (2012) J. Am. Stat. Assoc. 107, 1590-1598) has been used.

In yet another embodiment the invention provides an in vitro method for predicting the response to a percutaneous coronary intervention (PCI) by quantifying the extent of functional CAD.

In yet another embodiment the invention provides a method to select a mammal suffering from coronary artery disease (CAD) to be eligible for a percutaneous coronary intervention (PCI) comprising the application of the computer-implemented method according to the methods herein described and selecting a mammal when the extent of functional disease in the coronary artery is smaller than the extent of anatomical disease in the coronary artery.

In a particular embodiment the mammal is eligible for a PCI when the functional-anatomical mismatch (FAM) is higher than 0 (FAM=>0). This means that the length of the functional CAD is described within the anatomical defined CAD.

In another embodiment the mammal is not eligible for a PCI (or not eligible for having a successful PCI procedure) when the FAM is lower than 0 (FAM=<0). This means that the length of the functional CAD extends beyond the anatomical defined lesion. This means that a negative FAM value reflects to pressure losses outside the determined anatomical lesion.

In specific embodiments the methods are offline methods.

In other specific embodiments the methods are automatic classification methods.

In yet another embodiment the invention provides a computer device for evaluating the functional pattern of coronary artery disease in a mammal, said computer device configured to process a set of FFR values obtained at different positions of the coronary vessel between the ostium and the most distal part of the coronary vessel and classifying the coronary vessel in focal and/or diffused diseased segments by carrying out a piece-wise linearization of said FFR data by applying an automated change-points detection algorithm based on a penalized parametric global method.

In a specific embodiment the set of FFR values are obtained from a pull-back curve, 3-dimensional quantitative coronary angiography, CT scan or optical coherence tomography (OCT).

The methods of the invention can also be used in veterinary applications. Mammals comprise cats, dogs, horses, cows, goats, sheep and preferably human subjects (human patients).

In other specific embodiment the fractional flow reserve (FFR) data are obtained by a manual or motorized pullback device which device is attached to the pressure wire.

In yet another particular embodiment there is no need for a motorized pullback device but instead the FFR curve is obtained by a pressure wire comprising a multiple of built-in pressure sensors.

In another particular embodiment, the catheter is configured to obtain diagnostic information about the coronary vessel. In this respect, the catheter can include one or more sensors, transducers, and/or other monitoring elements configured to obtain the diagnostic information about the vessel. The diagnostic information includes one or more of pressure, flow (velocity), images (including images obtained using ultrasound (e.g., intravascular ultrasound—IVUS), optical coherence tomography (OCT), thermal, and/or other imaging techniques), temperature, and/or combinations thereof. These one or more sensors, transducers, and/or other monitoring elements are positioned less than 30 cm, less than 10 cm, less than 5 cm, less than 3 cm, less than 2 cm, and/or less than 1 cm from a distal tip of the catheter in some instances. In some instances, at least one of the one or more sensors, transducers, and/or other monitoring elements is positioned at the distal tip of the catheter. In another particular embodiment the catheter comprises at least one element configured to monitor pressure within the coronary vessel. The pressure monitoring element can take the form a piezo-resistive pressure sensor, a piezo-electric pressure sensor, a capacitive pressure sensor, an electromagnetic pressure sensor, an optical pressure sensor, and/or combinations thereof. In some instances, one or more features of the pressure monitoring element are implemented as a solid-state component manufactured using semiconductor and/or other suitable manufacturing techniques.

In yet another embodiment the catheter comprises a pressure wire (or a guide wire). Examples of commercially available guide wire products that include suitable pressure monitoring elements include, without limitation, the Prime Wire PRESTIGE® pressure guide wire, the Prime Wire® pressure guide wire, and the ComboWire® XT pressure and flow guide wire, each available from Volcano Corporation, as well as the Pressure Wire™ Certus guide wire and the Pressure Wire™ Aeris guide wire, each available from St. Jude Medical, Inc or COMET™ FFR pressure guidewire from Boston Scientific. The pressure wire is also configured to obtain diagnostic information about the coronary vessel. In some instances, the pressure wire is configured to obtain the same diagnostic information as the catheter. In other instances, the pressure wire is configured to obtain different diagnostic information than the catheter, which may include additional diagnostic information, less diagnostic information, and/or alternative diagnostic information. The diagnostic information obtained by the pressure wire includes one or more of pressure, flow (velocity), images (including images obtained using ultrasound (e.g. IVUS), OCT, thermal, and/or other imaging techniques), temperature, and/or combinations thereof.

Similar to the catheter the pressure wire also includes at least one element configured to monitor pressure within the vessel. The pressure monitoring element can take the form a piezo-resistive pressure sensor, a piezo-electric pressure sensor, a capacitive pressure sensor, an electromagnetic pressure sensor, an optical pressure sensor, and/or combinations thereof. In some instances, one or more features of the pressure monitoring element are implemented as a solid-state component manufactured using semiconductor and/or other suitable manufacturing techniques. In a particular embodiment the pressure wire can comprise multiple pressure sensors, e.g. at least 10, at least 20, at least 30, at least 40, at least 50, or more pressure sensors. It is clear that according to such embodiments of the pressure wire, the multiple pressure sensors are provided at different positions along the length of the pressure wire, and thus configured to, even when stationary, after being introduced into the coronary vessel up to the distal end of the coronary vessel, determine a plurality of pressure measurements at different positions along the length of the coronary vessel, or in other words at different positions between the ostium and the distal end of the coronary vessel.

In a particular embodiment the pressure wire is configured to monitor pressure within the vessel while being moved through the lumen of the vessel. In some instances, the pressure wire is configured to be moved through the lumen of the vessel and across the stenosis present in the vessel. In that regard, the pressure wire is positioned distal of the stenosis and moved proximally (i.e. pulled back) across the stenosis to a position proximal of the stenosis in some instances. Movement of the pressure wire can be controlled manually by medical personnel (e.g. hand of a surgeon) in some embodiments. In other preferred embodiments, movement of the pressure wire is controlled automatically by a movement control device (e.g. a pullback device, such as the Trak Back® II or Volcano R-100 Device available from Volcano Corporation). In that regard, the movement control device controls the movement of the pressure wire at a selectable and known speed (e.g. 5.0 mm/s, 2.0 mm/s, 1.0 mm/s, 0.5 mm/s, etc.) in some instances. Movement of the pressure wire through the vessel is continuous for each pullback, in some instances. In other instances, the pressure wire is moved step-wise through the vessel (i.e. repeatedly moved a fixed amount of distance and/or a fixed amount of time).

In yet another embodiment the invention provides a system for evaluating coronary artery disease in a patient under hyperaemic conditions, comprising i) a coronary catheter comprising a pressure sensor, said catheter further comprising a pressure wire comprising at least one pressure sensor, ii) a computing device in communication with the catheter and the pressure wire, the computing device configured to generate an FFR curve based on a multiple of FFR values (the latter are relative pressure measurements from pressures obtained over the total length of the coronary vessel relative to the pressure in the ostium), iii) said computer device comprising a computer algorithm which calculates the length (or the extent) of the a functional coronary disease based on the FFR curve, and the correlation with the anatomical coronary artery disease, the computer output displays an FAM value which informs an interventional cardiologist of a successful percutaneous coronary intervention based on the positive or negative value of the FAM value. In particular, when the FAM value is negative (i.e. <0) there is no likelihood of conducing a successful PCI on the patient.

In the present invention a “system” is equivalent to a “device” or an “apparatus”. It is clear that such a system, device and/or apparatus could comprise any suitable number of interconnected subsystems or components which could be housed in the same housing or in a plurality of different housings.

A computing device is generally representative of any device suitable for performing the processing and analysis techniques discussed within the present disclosure. In some embodiments, the computing device includes a processor, random access memory, and a storage medium. In that regard, in some particular instances the computing device is programmed to execute steps associated with the data acquisition and analysis described herein. Accordingly, it is understood that any step related to data acquisition, data processing, calculation of the FAM, instrument control, and/or other processing or control aspects of the present disclosure may be implemented by the computing device using corresponding instructions stored on or in a non-transitory computer readable medium accessible by the computing device. In some instances, the computing device is a console device. In some instances, the computing device is portable (e.g. handheld, on a rolling cart, etc.). Further, it is understood that in some instances the computing device comprises a plurality of computing devices. In that regard, it is particularly understood that the different processing and/or control aspects of the present disclosure may be implemented separately or within predefined groupings using a plurality of computing devices. Any divisions and/or combinations of the processing and/or control aspects described herein across multiple computing devices are within the scope of the present disclosure.

It is understood that any communication pathway between the catheter and the computing device may be utilized, including physical connections (including electrical, optical, and/or fluid connections), wireless connections, and/or combinations thereof. In that regard, it is understood that the connection is wireless in some instances. In some instances, the connection a communication link over a network (e.g. intranet, internet, telecommunications network, and/or other network). In that regard, it is understood that the computing device is positioned remote from an operating area where the catheter is being used in some instances. Options for the connection include a connection over a network which can facilitate communication between the catheter and the remote computing device regardless of whether the computing device is in an adjacent room, an adjacent building, or in a different state/country. Further, it is understood that the communication pathway between the catheter and the computing device is a secure connection in some instances. Further still, it is understood that, in some instances, the data communicated over one or more portions of the communication pathway between the catheter and the computing device is encrypted.

### DISCUSSION AND SUMMARY

The present invention provides a computer device and a computer-implemented method for the quantification of the extension of functional coronary artery disease (CAD) in a mammal, such as a human patient. It is clear that the extension of the functional CAD corresponds to the functional length of the CAD. The method determines the mismatch in the extent of CAD between anatomical and physiological invasive evaluations based on angiography, intravascular imaging and intracoronary hyperemic pressure tracing pullbacks. In particular, the extent of functional disease derived from FFR data (such as FFR data derived from pullback curves) can be quantified using a specially developed algorithm provided herein. The clinical relevance of the methods provided is that the mismatch between the length of anatomical and functional CAD (i.e. abbreviated as FAM, either derived from QCA or OCT) predicts improvement in epicardial conductance after percutaneous revascularization. It is clear that the length or extent of the anatomical CAD is determined by means of a detection of a particular part of the vessel comprising a reduction of the diameter, or the lumen area of the coronary vessel, or any other suitable indicator of an anatomical diversion of the vessel which for example exceeds a predetermined threshold. It is clear that the length or extent of the anatomical CAD is correlated to the part of the vessel, which can be considered as anatomically diseased as its anatomy impacts the blood flow along the coronary vessel negatively. It is clear that the length or extent of the functional CAD is determined by means of a detection of particular linearized segments of the vessel correlating to a reduction in the FFR values, or any other suitable indicator of a pressure change, which exceeds a predetermined threshold, and thereby determines the extent or length of the vessel which can be considered as functionally diseased based on the fact that, irrespective of detectable anatomical indicators, the functionality of the coronary artery in these segments is negatively affected.

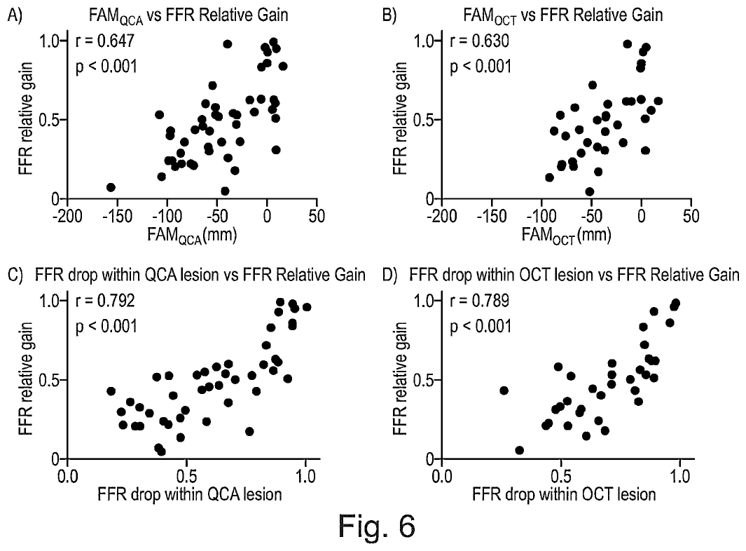

QCA is based on conventional angiography and identifies CAD length as the extent of the stenotic segment. It is clear that, as described above, this refers to the extent or length of the anatomical CAD, which according to this embodiment is determined by means of QCA. On the other hand, OCT, possessing higher spatial resolution, derives lesion length from the selection of proximal and distal reference cross-sections without atherosclerotic plaques. It is clear that, this refers to the extent or length of the anatomical CAD, which according to this embodiment is determined by means of OCT. Therefore, it is expected that embodiments with CAD anatomical length derived from OCT will be equal or longer than embodiments with the QCA-derived length. We observed that the anatomical length of CAD was shorter when derived from QCA compared to the one derived from OCT; still, FFR pullbacks derived CAD length was longer (FIG. 6). In other words, according to this embodiment, the functional length of the CAD was longer than both embodiments of the anatomical length of the CAD. This finding highlights the diffuse nature of atherosclerosis when assessed using coronary physiology. Interestingly, CAD extent derived from QCA and functional length were not statistically correlated whereas OCT-derived anatomical length and functional length exhibited a moderate correlation. Our data underline the suboptimal guidance offered by QCA in terms of the evaluation of lesion extension and confirms the usefulness of intravascular imaging for attaining functional complete revascularization. In other words, when taking only into account the QCA derived anatomical extent or length of the CAD, suboptimal guidance in terms of evaluation of lesion extension is offered.

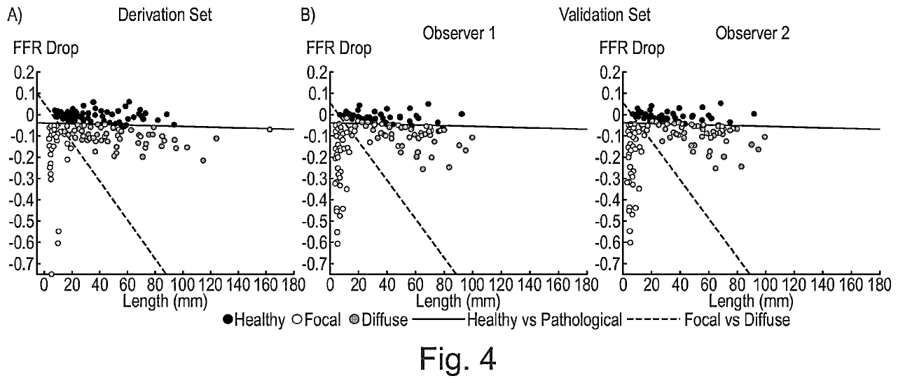

Pressure pullbacks can show two distinct functional CAD endotypes, namely predominant focal or diffuse. In the focal functional CAD, pressure drops are commonly restricted to anatomical stenosis. In this disease endotype, PCI restores epicardial conductance, results in higher post-PCI FFR, increases the likelihood of relieving patients from angina and is associated with improved clinical outcomes. In contrast, in patients with functional diffuse disease, PCI results in minor improvement in vessel physiology, low post-PCI FFR and higher likelihood of persistent angina. Several novel methods are available to assess the pattern of CAD aiming at predicting the results of PCI in terms of coronary physiology. The pullback pressure gradient (PPG) index (Coroventis Research, Uppsala, Sweden), instant wave-free ratio (iFR) co-registration system (Philips, Best, the Netherlands) and the FFR_{CT} revascularisation planner (HeartFlow Inc, Redwood city, USA) are novel approaches that may further personalize clinical decision making and refine revascularization strategies in patients with chronic coronary syndromes. In the present invention, we developed a complementary approach to predict the response to PCI by quantifying the extent of functional CAD from FFR pullbacks. The larger the functional length of CAD, the lower the functional gain obtained with PCI and the lower the likelihood of functional revascularization. This approach is analogous to the PPG where millimeters with functional disease are calculated based on an FFR threshold. In other words, according to this embodiment, there is defined a threshold of for example an FFR drop ≥0.0015/mm for labeling the parts of the coronary vessel exhibiting FFR deterioration. It is clear that, according to this embodiment, similarly this threshold, defines the parts of the coronary vessel which do not exhibit FFR deterioration. In the context of the PPG index, as referred to above, the length or extent of the functional disease was derived from a pullback curve, by for example aggregating the length of all parts of the curve where the FFR drop, or in other words the FFR reduction was ≥0.0015/mm. According to the embodiment of the current approach, as described above, first the set of FFR values, for example representing an FFR pullback curve, are transformed by means of the piece-wise linearization by applying an automated change-points detection algorithm into a sequency of segments, such as for example healthy segments and diseased segments, for example comprising focal diseased segments, diffused diseased segments, or any other suitable diseased segments. With the current approach, the length of functional disease is computed based on an automated algorithm classifying the FFR curve segments as healthy or diseased. In other words, after processing the set of FFR values, representing for example an FFR pullback curve, the automated change-points detection algorithm, converts the set of FFR values, by means of piece-wise linearization, into a sequence of linear segments, which are classified as healthy segments or diseased segments. According to a preferred embodiment, such a segment is classified as a healthy segment, when the segment does not exhibit FFR deterioration, or in other words, when for example the FFR drop and segment length of the segment define a position in a coordinate system, in which the FFR drop is the y-axis and in which the segment length is the x-axis, which is above a predetermined first classification threshold function. It is clear that the FFR drop is defined as the difference between the FFR values at the distal and at the proximal point of the segment. According to the exemplary embodiment shown in FIG. 4 by means of the full line labeled “Healthy vs Pathological”, the predetermined first classification threshold function is for example expressed by means of the following equation, y=(−1,6536.10⁻⁴). x−0.0393, in which y is the value for the FFR drop and x is the segment length in mm. It is clear that, according to the embodiment shown, any segment with a corresponding x, y coordinate determined by respectively the segment length and FFR drop of that segment, positioned above that first classification threshold function, according to this embodiment, is classified as a healthy segment, and represented by means of a black marker. According to a preferred embodiment, such a segment is classified as a diseased segment, when the segment does exhibits FFR deterioration, or in other words, when the FFR drop and segment length of the segment define a position in a coordinate system in which the FFR drop is the y-axis and in which the segment length is the x-axis, which is below the predetermined first classification threshold function. According to the exemplary embodiment shown in FIG. 4, the predetermined first classification threshold function is for example expressed by means of the following equation, y=(−1,6536.10⁻⁴). x−0.0393, in which y is the value for the FFR drop and x is the segment length in mm. It is clear that, according to the embodiment shown, any segment with a corresponding x, y coordinate determined by respectively the segment length and FFR drop of that segment, positioned below that first classification threshold function, is classified as a diseased segment, and represented by means of a white or gray markers. It is however clear that this first classification threshold function according to this embodiment was derived from a particular set of patient data, and it is clear that when the classification is based on alternative and/or additional patient data other suitable classification threshold functions may be derived. In other words, according to alternative embodiments, any suitable first classification threshold function, configured to classify healthy segments and diseased fragments, based on suitable parameters of the segment, such as the FFR drop and/or segment length, and/or any suitable ration, or combination thereof of these segments is possible. Another advantage of our approach is that it is less vulnerable to artefacts in the pullback curves compared to the application of a threshold without making use of piece-wise linearization by means of an automated change point detection algorithm. It is clear that piece-wise linearization by means of an automated change point detection algorithm, by means of the parameters determined based on the linear segments is less sensitive to local artefacts, which are for example the result of temporary or local measurement errors, etc. Moreover, the FAM concept by incorporating the anatomical length of the disease accounts for the interaction with PCI, thus assessing the functional contribution in the context of the segment to be treated. Revascularization aims at restoring epicardial conductance to improve myocardial perfusion. Two factors influence the response of epicardial vessels to PCI in terms of coronary physiology. First, the functional contribution of the treated lesion to the complete vessel pressure loss. In other words, the magnitude of the FFR curve step-up within the lesion relative to the overall reduction of FFR in the vessel. Second, the presence of pressure losses outside the treated segment. FAM quantifies the mismatch between the anatomical and functional CAD length thereby stressing the impact of residual pressure losses outside the treated region on post PCI physiology. Moreover, the FAM approach is based on the presence and length of disease rather than on the magnitude of pressure drops making this approach less influenced by the interaction in cases of serial lesions. Furthermore, the functional contribution of the treated lesion, shown in this invention as relative FFR drop within the lesion, also correlated with functional gain (FIGS. 6, C and D). The larger the delta lesion FFR, the larger the functional gain. Altogether, our findings further support pressure pullback strategies to guide PCI as a second level of decision making after the confirmation of hemodynamic lesion significance. By identifying lesions with negative FAM where the functional extent of CAD is longer than the anatomical extent of CAD, a dilemma is posed upon clinicians. Extending the treated region with longer stents covering the functional disease may improve post-PCI FFR but it may also lead to longer and more stents which has been associated with higher rates of target vessel failure. Moreover, PCI in vessels with functional diffuse disease has been associated with more periprocedural complications. Therefore, the assessment of the functional pattern of CAD provides further risk stratification, may improve patient selection for PCI by avoiding stenting lesions without pullback step ups, reduce the risk of peri-procedural myocardial infarction and results in a net clinical benefit from revascularization. With the methods of the instant invention we foresee that patients with a negative FAM, i.e. having diffuse functional CAD, may be better treated with optimal medical therapy or coronary artery bypass grafting whereas patients with a positive FAM are better treated with PCI.

It is also evident that the information obtained regarding characteristics of the coronary artery disease, such as for example the measurement of the length of the anatomical and functional region, can be considered in addition to other representations of the lesion or stenosis and/or the vessel, such as e.g. IVUS, for example including virtual histology, OCT, ICE, Thermal, Infrared, flow, Doppler flow, and/or other vessel data-gathering modalities, to provide a more complete and/or accurate understanding of the vessel characteristics. For example, in some instances the information regarding characteristics of the lesion or stenosis and/or the vessel as obtained by the system of the invention are utilized to confirm information calculated or determined using one or more other vessel data-gathering modalities.

Finally, it is to be understood that although particular embodiments, specific configurations as well as materials and/or molecules, have been discussed herein for methods according to the present invention, various changes or modifications in form and detail may be made without departing from the scope of this invention. The following examples are provided to better illustrate particular embodiments, and they should not be considered limiting the application. The application is limited only by the claims.

### Examples

### 1. Patient Characteristics

Clinical characteristics of patients are shown in Table 1. Overall, 117 patients (131 vessels) were included: 71 patients (81 vessels) in the derivation cohort and 48 patients (50 vessels) in the validation cohorts. QCA and OCT anatomical lesion lengths were available for all (n=50) and part (n=36) of the validation cohort, respectively (Table 2). FFR motorized pullbacks pre and post PCI were available in all cases.

### 2. FFR Pullback Curve Automatic Classifier

Anatomical, functional, and procedural characteristics of the derivation and validation cohort are presented in Table 2. From the FFR curves, 431 segments were extracted. In detail, 151 (observer 1) and 156 (observer 2) segments were visually assessed as healthy, 101 (observer 1) and 106 (observer 2) as focal disease, 146 (observer 1) and 147 (observer 2) as diffuse disease (FIG. 4). In the validation cohort, the automatic classifier provided an excellent discrimination ability between ‘healthy’ and ‘diseased’ segments (AUC was 0.97, 95% CI 0.94 to 0.99 for observer 1, and 1.00, 95% CI 0.98 to 1.00 for observer 2). Concerning differentiation between focal and diffuse diseased segments, the discriminatory capacity was also excellent (AUC was 0.93, 95% CI 0.86 to 0.97 for observer 1, and 0.96, 95% CI 0.91 to 0.99 for observer 2). The inter-observer agreement for the visual evaluation of the FFR curve was moderate (ICC 0.66, 95% CI 0.60 to 0.71).

### 3. Functional Anatomical Mismatch (FAM)

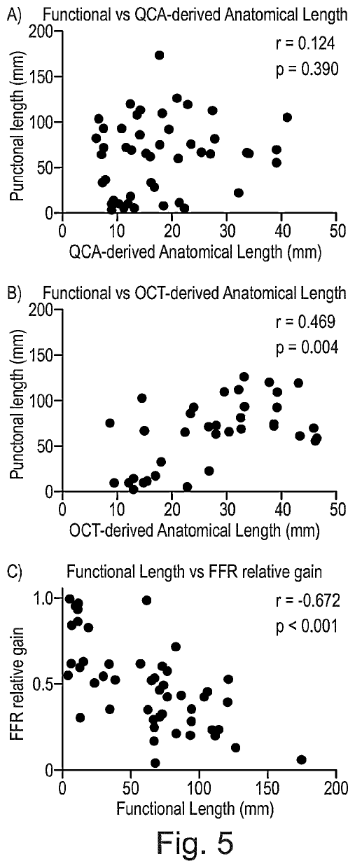

PCI was performed in 50 vessels included in the validation cohort. Pre-PCI FFR was 0.74 [0.67-0.77] and diameters stenosis was 53.0% [47.25-59.50]. Anatomical CAD length derived from QCA was 16.05 mm [11.40-22.05], anatomical CAD length derived from OCT was 28.0 mm [16.63-38.0] and functional CAD length was 67.12 mm [25.38-91.37] (p<0.001). No correlation emerged between the extent or length of anatomical CAD derived from QCA and the extent or length of functional CAD derived from FFR pullbacks (r=0.124, 95% CI 0.168 to 0.396, p=0.390, FIG. 5A). OCT-derived anatomical lesion length was correlated with functional CAD length (r=0.469, 95% CI 0.156 to 0.696, p=0.004, FIG. 5B).

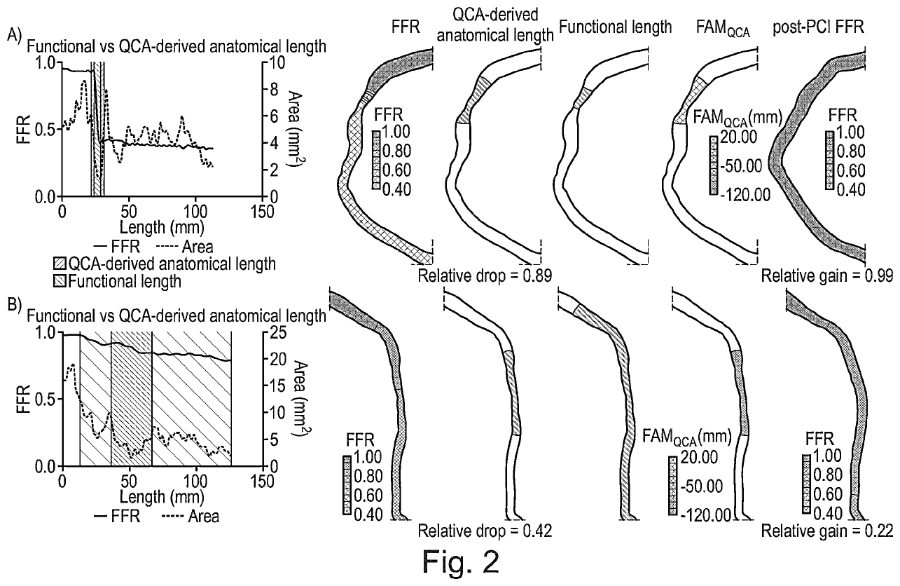

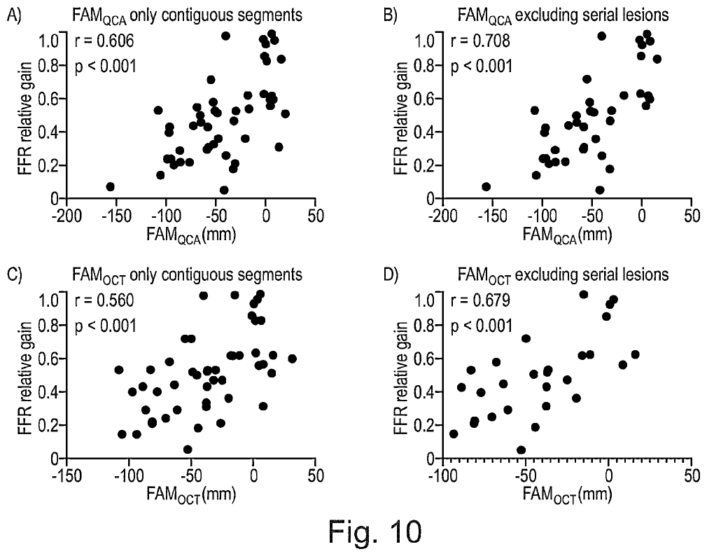

Mean stent length was 27.45±11.52 mm. Mean post-PCI FFR was 0.86 [0.82-0.89]. An explanatory example visualizing vessels with positive and negative FAM that underwent PCI and post-PCI FFR measurement is presented in FIG. 2. FAM_{QCA} was −47.59 [−73.22-−8.08] mm and FAM_{OCT} was −37.47 [−64.29-−8.98] mm (p=0.446). The length of functional CAD was inversely correlated with relative functional gain (r=−0.672, 95% CI−0.804 to −0.478, p<0.001, FIG. 5C) i.e., the longer the functional disease the lesser the improvement in FFR with PCI. A strong association emerged between FAM_{QCA} and FFR relative gain after PCI (r=0.647, 95% CI 0.443 to 0.788, p<0.001, FIG. 6A), as well as between FAM_{OCT} and FFR relative gain after PCI (r=0.630, 95% CI 0.372 to 0.798, p<0.001, FIG. 6B). A sensitivity analysis in the sub-group of serial lesions showed similar results (FIG. 10). It is clear that FFR gain after PCI corresponds to the difference of the vessel FFR after PCI with respect to the vessel FFR before PCI. It is clear that the vessel FFR refers to the FFR calculated from the distal pressure or Pd at the most distal part of the coronary artery, with respect to the proximal or aortic pressure Pa at the ostium of the coronary artery.

Patients in whom functional disease was confined within the anatomical lesion (i.e. FAM≥0) had the strongest improvement in relative functional gain (FAM_{QCA}≥0.701±0.235 vs. FAM_{QCA}<0 0.441±0.225, p<0.001). FAM either derived from QCA or OCT predicted functional gain (FAM_{QCA} AUC 0.84, 95% CI 0.71 to 0.93, p<0.001 and FAM_{OCT} AUC 1.00, 95% CI 0.93 to 1.00, p<0.001). The best FAM_{QCA} and FAM_{OCT} cutoff values predicting 50% gain in epicardial conductance were −57.64 mm and −37.19 mm, respectively. Percent FFR drops within the anatomical lesions either derived from QCA or OCT were strongly correlated with functional gain (r=0.792, 95% CI 0.655 to 0.879, p<0.001 for QCA and r=0.789, 95% CI 0.615 to 0.890, p<0.001 for OCT, FIG. 6C-6D).

Tables

TABLE 1
Baseline clinical characteristics

| | | | Derivation | | Validation | |
|---|---|---|---|---|---|---|
| | All | | Cohort | | Cohort | |
| Patients, n | 117 | | 69* | | 48 | |
| Vessels, n | 131 | | 81 | | 50 | |
| Age, mean ± SD | 66.9 ± 9.84 | | 68.2 ± 9.6 | | 64.7 ± 9.9 | |
| Sex, male, n (%) | 86 | (73.5) | 52 | (73.2) | 36 | (75.0) |
| BMI, kg/m², mean ± SD | 26.8 ± 3.53 | | 26.7 ± 3.52 | | 26.8 ± 3.32 | |
| Hyperlipidemia, n (%) | 94 | (80.3) | 57 | (80.3) | 38 | (79.2) |
| Hypertension, n (%) | 63 | (53.8) | 36 | (50.7) | 29 | (60.4) |
| Diabetes Mellitus, n (%) | 26 | (22.2) | 17 | (23.9) | 9 | (18.8) |
| Current smoking, n (%) | 20 | (17.1) | 12 | (16.9) | 9 | (18.8) |
| Family history, n (%) | 17 | (14.5) | 10 | (14.1) | 8 | (16.7) |
| Previous stroke, n (%) | 3 | (2.6) | 3 | (4.2) | 0 | (0) |
| Prior PCI, n (%) | 32 | (27.4) | 28 | (39.4) | 4 | (8.3) |
| LVEF, %, mean ± SD | 58.0 ± 8.06 | | 58.1 ± 9.28 | | 58.0 ± 5.57 | |
| Creatinine, mg/dl, mean ± SD | 0.98 ± 0.22 | | 0.99 ± 0.24 | | 0.96 ± 0.18 | |
| Symptomatic, n (%) | 91 | (77.8) | 50 | (70.4) | 43 | (89.6) |
| Angina Class, CCS | | | | | | |
| 1 | 26 | (28.6) | 15 | (30.0) | 11 | (25.6) |
| 2 | 61 | (67.0) | 32 | (64.0) | 31 | (72.1) |
| 3 | 4 | (4.4) | 3 | (6.0) | 1 | (2.3) |
| 4 | 0 | (0) | 0 | (0) | 0 | (0) |

*In two patients, two vessels were analyzed and one underwent PCI. For patient level characteristic these were included in the validation cohort.
PCI Percutaneous coronary interventions,
CCS Canadian classification society,
LVEF Left ventricular ejection fraction,
BMI Body mass index,
SD Standard deviation

TABLE 2
Anatomical, functional and procedural characteristics

| | | Derivation | | Validation | |
|---|---|---|---|---|---|
| Target vessel, n (%) | All | Cohort | | Cohort | |
| LAD | 99 (75.6) | 57 | (70.4) | 42 | (84.0) |
| LCX | 9 (6.9) | 6 | (7.4) | 3 | (6.0) |
| RCA | 23 (17.6) | 18 | (22.2) | 5 | (10.0) |
| Serial lesion, n (%) | 17 (13.0) | 9 | (11.1) | 8 | (16.0) |

| QCA-derived anatomical lesion length | NA | NA | 16.05 | |
|---|---|---|---|---|
| (mm), median [25th-75th percentile] | | | [11.40-22.05] | |
| OCT-derived anatomical lesion length | NA | NA | 28.00 | |
| (mm), median [25th-75th percentile] | | | [16.63-38.00] | |
| MLD (mm), median [25th-75th | NA | NA | 1.34 | |
| percentile] | | | [1.23-1.48] | |
| DS (%), median [25th-75th percentile] | NA | NA | 53.00 | |
| | | | [47.25-59.50] | |
| RVD (mm), median [25th-75th | NA | NA | 2.95 | |
| percentile] | | | [2.57-3.20] | |
| Functional lesion length (mm), median | NA | NA | 67.12 | |
| [25th-75th percentile] | | | [25.38-91.37] | |
| FAM_{QCA} (mm), median [25th-75th | NA | NA | −47.59 | |
| percentile] | | | [−73.22-−8.08] | |
| FAM_{OCT} (mm), median [25th-75th | NA | NA | −37.47 | |
| percentile] | | | [−64.29-−8.98] | |
| Pre PCI FFR, median [25th-75th | 0.78 | 0.82 | 0.74 | |
| percentile] | [0.71-0.86] | [0.73-0.88] | [0.67-0.77] | |
| PCI, number | NA | NA | 54 | |
| Stent per vessel, n (%) | NA | NA | 1.00 | |
| | | | [1.00, 1.00] | |
| Stent length (mm) | NA | NA | 27.45 ± | |
| | | | 11.52 mm | |
| Stent diameter (mm) | NA | NA | 3.05 ± 0.43 | |
| Post dilatation, n (%), | NA | NA | 46 | (85.2) |
| IVUS/OCT-guided PCI, n (%) | NA | NA | 42 | (84.0) |

| | | | |
|---|---|---|---|
| MSA (mm²), median [25th-75th | NA | NA | 5.66 |
| percentile] | | | [4.33-6.45] |
| Residual DS (%), median [25th-75th | NA | NA | 7.00 |
| percentile] | | | [2.00-12.00] |
| Acute gain | NA | NA | 1.34 ± 0.54 |
| Post PCI FFR, median [25th-75th | NA | NA | 0.86 |
| percentile] | | | [0.82-0.89] |
| Relative functional gain, median | NA | NA | 0.49 |
| [25th-75th percentile] | | | [0.30-0.62] |

### Methods

### 1. Study Design

This is a multicenter, prospective registry of patients undergoing clinically indicated coronary angiography in whom motorized FFR pullback evaluations were performed before PCI. Patients presenting with acute coronary syndromes, previous coronary artery bypass grafting, significant valvular disease, severe obstructive pulmonary disease or bronchial asthma, coronary ostial lesions, severe tortuosity, or severe calcification were excluded. Patients with adequate pressure tracings and pullback curves were included in this analysis. The study was approved by the Ethics Committee at each participating center. The study population is a combination of two prospective studies NCT03824600 and NCT03782688.

### 2. Coronary Angiographic Analysis

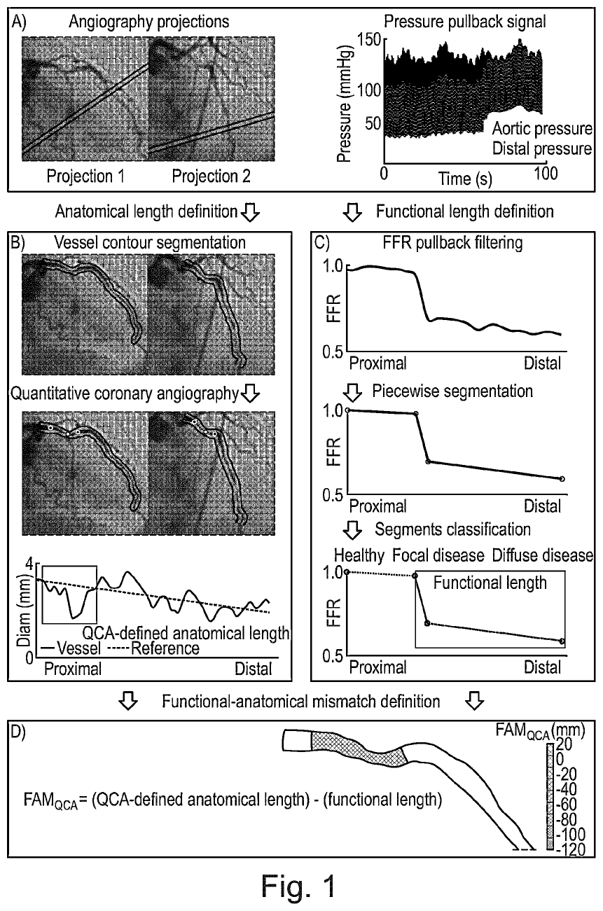

Angiographies were performed using a dedicated acquisition protocol. Two angiographic projections separated at least 30 degrees were obtained for each target lesion after the administration of intracoronary nitrates (FIG. 1A). Angiograms were evaluated blinded to physiological and clinical data and were analyzed using three-dimensional quantitative coronary angiography (3D QCA) (QAngio XA, Medis Medical Imaging, Netherlands). Minimal lumen diameter (MLD), reference vessel diameter (RVD), and percentage diameter stenosis (% DS) were calculated. Acute gain was defined as the difference between post and pre-PCI MLD. QCA-derived anatomical lesion length was calculated using the 3D QCA software and defined as the length where the reference diameter line intersects diameter function line (FIG. 1B). As referenced above, and as shown in FIG. 1B the QCA defined anatomical length is determined by the length of such sections where the reference diameter line intersects the diameter function line, or in other words the reference diameter line, and which, for example based on the minimal lumen diameter, are qualified by means of a suitable computer-implemented method as stenosis lesions, and selected for the calculation of the anatomical length. Manual correction of anatomical lesion length was not allowed. Serial lesions were defined as the presence of at least two visual diameter stenosis lesions within the same vessel, at a distance of at least three times the reference vessel diameter. As referenced above, it is however clear that according to alternative embodiments other suitable computer-implemented methods for determining the portions or sections of the vessel that qualify as an anatomical lesion, and of which the length is taken into account for calculating the anatomical lesion length are possible, such as for example based on a suitable threshold for the reduction of the diameter of the vessel with respect to diameter reference values are possible, such as for example at least 30%, or at least 50%, or any other suitable threshold.

### 3. Optical Coherence Tomography (OCT)

Examinations were performed using the OPTIS™ OCT systems (Abbott Vascular). OCT pullback at 36 mm/s were acquired before pre-dilation if feasible. OCT-derived anatomical lesion length was defined as the distance between the proximal and distal reference segments using the OCT automated lumen detection feature. Stent diameter selection was based on the distal reference mean external elastic lamina (EEL)-based diameters rounded down to the nearest available stent size (usually in 0.25 mm increments) to determine stent diameter. If the EEL could not be adequately visualized, the stent diameter is chosen using the mean lumen diameter at the distal reference rounded up to the next stent size. Optimization of the device for performed based on OCT at operator discretion.

### 4. Intracoronary Pressure Measurement and FFR Pullback Curve Analysis

Fractional flow reserve (FFR) measurements were performed with the Pressure Wire X (Abbott Vascular, Chicago, Il, USA) that was connected to a motorized pullback device at a speed of 1 mm/s (R 100, Philips Volcano, San Diego, Ca, USA). Pressure pullback measurements were acquired at a sampling frequency of 100 Hz. A continuous intravenous adenosine infusion was given at a dose of 140 mg/kg/min via a peripheral or central vein to obtain steady-state hyperemia for at least 2 min. The position of the pressure sensor was recorded with a contrast injection to identify the pullback initial position for co-registration purposes. In cases undergoing PCI, FFR measurements were repeated at the same anatomical location. FFR gain was defined as FFR post-minus FFR pre-PCI. If FFR drift (>0.03) was observed, the FFR pullback was repeated. For the FFR gain a FFR post and pre-PCI were determined as the ratio of the distal pressure or Pd at the most distal part of the coronary artery, with respect to the proximal or aortic pressure Pa at the ostium of the coronary artery.

The FFR curve along the vessel axis was reconstructed by applying a moving average filter with a window size of 10 s, followed by an infinite impulse response low pass elliptic filter (0.1 Hz cutoff frequency) for smoothing (FIG. 1C). It is clear that alternative embodiments are possible for smoothing the FFR pullback curve, or in other words the FFR data, such as for example any suitable moving average filter with a window size set to a suitable portion of the time period or length associated with the FFR pullback curve. According to still further embodiments, the window size of such a moving average filter could for example be set to a time period which corresponds to two, three, four, five, etc. or any other suitable number of heartbeat cycles, or in any other suitable way. It is clear that still further alternative embodiments for smoothing the FFR pullback curve are possible, in which for example use is made of a suitable moving average function, moving mean function, low pass filter, etc. in order to reduce the cyclic variation in the pressure values from which the FFR is derived during each cardiac cycle.

Such an embodiment is especially useful for quantifying the extent and/or patterns of coronary artery functional disease in a coronary vessel from a patient under hyperaemic conditions, wherein the patient is a mammal, for example a human. Under such conditions there can be generated pressure values that represent an FFR pullback curve during a pullback operation. Determining such an FFR pullback curve is done by determining FFR values from measurements of the movable pressure sensor, also referred to as distal pressure or Pd, with respect to a stationary pressure sensor, also referred to as proximal or aortic pressure Pa, during the pullback time period. It is clear that for example the stationary pressure sensor is positioned at the ostium of the vessel and that the movable pressure sensor, during the pullback time period is moved between a more distal part of the vessel, for example the most distal part of the vessel, or a part of the vessel distal of a suspected stenosis, stricture or lesion, and the ostium of the vessel. When such measurements are for example performed under hyperaemic conditions in a coronary artery, then these values that are determined based on these measurements during the pullback time period, during which the movable sensor is moved along the vessel, are referred to as FFR values of an FFR pullback curve, and are typically determined as the ratio of Pd/Pa, wherein Pd and Pa could for example be determined from the measured pressure values after any suitable form of pre-processing such as for example by means of a moving mean or average function which is configured to filter out the rhythmic and/or periodical component of the heartbeat cycle. It is thus clear that according to a preferred embodiment FFR could for example be defined as the ratio of mean or average distal coronary pressure, which is the pressure measured by the movable pressure sensor, and the mean or average aortic pressure, which for example is the pressure measured by the stationary pressure sensor, measured during, preferably maximal, hyperaemia that is preferably achieved through administration of a potent vasodilator such as for example adenosine, ATP or papaverine either by IV infusion or by intracoronary (IC) bolus injection. Under such conditions of hyperemia, by rendering myocardial microvascular resistance constant and minimal, the impact of disease in the epicardial conduit artery on myocardial blood flow is more advantageously separated out.

An automatic algorithm was developed for functional length quantification from FFR curves. The first step of the algorithm consisted in the piece-wise linearization of each FFR curve, see for example FIG. 1C, by applying an automated change-points detection algorithm based on a penalized parametric global method, as detailed in the next section. The second step of the algorithm consisted in the automatic classification of the linearized FFR curve segments as ‘healthy’ segments, i.e. without FFR deterioration, ‘focal’ or ‘diffuse’ disease segments, see for example FIG. 1C, based on their length and the associated FFR drop defined as the difference between the FFR values at the distal and at the proximal point of the segment. As described above, according to a preferred embodiment, such a segment is classified as a healthy segment, when the segment does not exhibit FFR deterioration, or in other words, when, for example the FFR drop and segment length of the segment define a position in a coordinate system, in which the FFR drop is the y-axis and in which the segment length is the x-axis, that is above a predetermined first classification threshold function. According to the exemplary embodiment shown in FIG. 4, the predetermined first classification threshold function is for example expressed by means of the following equation, y=(−1,6536.10⁻⁴). x−0.0393, in which y is the value for the FFR drop and x is the segment length in mm, however it is clear that alternative embodiments are possible, in which for example an alternative classification threshold function is determined based on a suitable dataset. According to a preferred embodiment, such a segment is classified as a diseased segment, when the segment does exhibits FFR deterioration, or in other words, when the FFR drop and segment length of the segment define a position in a coordinate system in which the FFR drop is the y-axis and in which the segment length is the x-axis, which is below the predetermined first classification threshold function. According to a preferred embodiment, such a segment is classified as a diffuse diseased segment, when the segment does exhibits FFR deterioration, or in other words, when the FFR drop and segment length of the segment define a position in a coordinate system in which the FFR drop is the y-axis and in which the segment length is the x-axis, which is below the predetermined first classification threshold function referenced above, such as for example y=(−1,6536.10⁻⁴). x−0.0393, and when the FFR drop and segment length of the segment define a position in a coordinate system in which the FFR drop is the y-axis and in which the segment length is the x-axis, which is above a predetermined second classification threshold function. According to the exemplary embodiment shown in FIG. 4 by means of the striped line labeled “Focal vs Diffuse”, the second classification threshold function is for example expressed by means of the following equation, y=(−0.0091). x+0.0575, in which y is the value for the FFR drop and x is the segment length in mm, however it is clear that alternative embodiments are possible, in which for example an alternative classification threshold function is determined based on a suitable dataset. According to a preferred embodiment, such a segment is classified as a focal diseased segment, when the segment does exhibit FFR deterioration, or in other words, when the FFR drop and segment length of the segment define a position in a coordinate system in which the FFR drop is the y-axis and in which the segment length is the x-axis, which is below the predetermined first classification threshold function referenced above, such as for example y=(−1,6536.10⁻⁴). x−0.0393, and when the FFR drop and segment length of the segment define a position in a coordinate system in which the FFR drop is the y-axis and in which the segment length is the x-axis, which is below the predetermined second classification threshold function. According to the exemplary embodiment shown in FIG. 4, the second classification threshold function is for example expressed by means of the following equation, y=(−0.0091). x+0.0575, in which y is the value for the FFR drop and x is the segment length in mm, however it is clear that alternative embodiments are possible, in which for example an alternative classification threshold function is determined based on a suitable dataset. It is clear that according to alternative embodiments, any suitable first classification threshold function configured to classify healthy segments versus diseased segments, and any suitable second classification threshold function configured to classify diffuse diseased segments versus focal diseased segments are possible, and these functions do not necessarily need to be linear function, but could be any other suitable continuous or discontinuous function.

Two cohorts were defined to develop and validate the part of the algorithm performing automatic FFR segments classification. The derivation cohort consisted of patients with CAD defined as distal FFR<0.90. For this cohort, only baseline (i.e. pre-PCI) FFR pullbacks were included. These were selected in a consecutive fashion from all patients included in the registry. The validation cohort included subsequent patients with CAD defined as a distal FFR 0.80 who underwent OCT-guided PCI and FFR measurement after stent implantation.

Two independent observers (observer 1: CaC; observer 2: SN) preliminarily adjudicated by visual inspection each one of the piece-wise linearized FFR curve segments belonging to the two cohorts as ‘healthy’, i.e. without FFR deterioration, or as ‘diseased’. Then, the two observers performed a further adjudication on ‘diseased’ segments, discriminating between ‘focal’ or ‘diffuse’, based on the presence of step-ups in the FFR pullback linearized curve.

The visual adjudication of the derivation cohort was used to develop the automatic classifier, based on a two-variables logistic regression. The two independent variables considered for the logistic regression were the length of the linearized segment and the associated FFR drop.

### 5. Automatic Segments Classification Method

### 5.1 Change Points Detection on FFR Pullback Curves

The detection of main changes in the distributional properties of FFR pullback curves was here addressed implementing a change points identification strategy. The implemented approach leads to a piece-wise linearization of FFR pullback curves based on a change points detection problem, where a change point is defined as a sample of the acquired FFR pullback curve at which an attribute of the curve suddenly changes. It is clear that a sample of the acquired FFR pullback curve, corresponds to a particular position along the part of the coronary vessel where the corresponding FFR pullback curve was generated. It is clear that a sudden change of the attribute of the FFR pullback curve, corresponds to an identifiable change, at that particular position in a relevant attribute of the FFR pullback curve, such as for example described in further detail below. As for example described below, the attribute of the FFR pullback curve, could for example be the average value and/or the slope along segments of the FFR pullback curve, or in other words subsets of the set of FFR values obtained at different positions of a coronary vessel between the ostium and the most distal part of the coronary vessel. Technically, a parametric global method detailed in Killick, R. et al (2012) J. Am. Stat. Assoc. 107, 1590-1598 and Lavielle M. (2005) Signal Processing 85, 1501-1510 were implemented here in MATLAB environment (MathWorks, Natick, MA, US) for FFR pullback change points identification. The steps of the implemented algorithm leading to a single change point detection are the following:
1. the FFR pullback curve is divided into two segments,
2. on each segment, the empirical estimation of the statistical property of interest is computed,
3. on each point of each segment the deviation from the empirical estimation is computed,
4. the total residual error is obtained by summation of deviations of segments points,
5. the location of the change point is identified iteratively minimizing the cost function represented by the total residual error.

It is thus clear that the change point is configured to divide the FFR pullback curve in two segments, in which the change point defines the endpoint between these two segments.

The problem expressed by points 1-5 can be translated into an algorithm as explained in the followings. Given a generic FFR pullback curve, FFR=(FFR₁, FFR₂, . . . , FFR_{N}), where FFRᵢ is the FFR value at i-th sample of the curve and N the total number of samples, the problem consists in finding the k-th sample minimizing the cost function

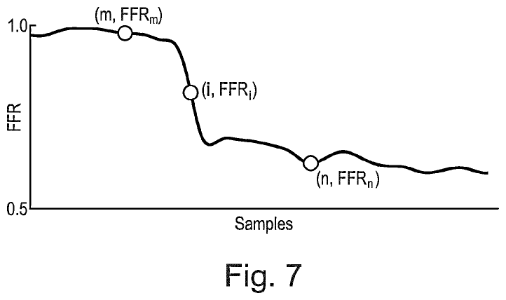

J(k)=Σᵢ₌₁^{k-1}Δ(FFRᵢ;χ([FFR₁ . . . FFRₖ₋₁]))+Σᵢ₌ₖ^{N}Δ(FFRᵢ;χ([FFRₖ . . . FFR_{N}])) (1),

where χ is the empirical estimation of the statistical property of interest and Δ is the deviation measure. Since we are interested in highlighting changes in average value and slope along the FFR pullback curve, here a linear function was adopted as statistical property of interest. This is like to say that for a generic interval between points m and n along the FFR pullback curve (FIG. 7), the terms at the right-hand side of Equation (1) can be expressed as:

$$( 2 )$$

$${\sum }_{i = m}^{n} Δ ( {FFR}_{i} ; 𝒳 ( [ {FFR}_{m} ... {FFR}_{n} ] ) ) == ( n - m + 1 ) var ( [ {FFR}_{m} ... {FFR}_{n} ] ) - \frac{\begin{matrix}( {\sum }_{i = m}^{n} ( {FFR}_{i} ; μ ( [ {FFR}_{m} ... {FFR}_{n} ] ) ) ) ( i - \\ {μ ( [ {FFR}_{m} {FFR}_{m + 1} ... {FFR}_{n} ] ) ) )}^{2}\end{matrix}}{( n - m + 1 ) var ( [ {FFR}_{m} {FFR}_{m + 1} ... {FFR}_{n} ] )}$$

A generic FFR pullback curve might have several change points, the number of change points being unknown a priori. Since adding change points decreases the residual error, the overfitting of the FFR curve is avoided by adding a penalty term which is a linear function of the number of change points to the cost function, which can be expressed as:

$$J ( C ) = {\sum }_{r = 0}^{C} {\sum }_{i = {k}_{r}}^{{k}_{r + 1} - 1} Δ ( {x}_{i} ; 𝒳 ( [ {FFR}_{{k}_{r}} ... {FFR}_{{k}_{r + 1} - 1} ] ) ) + β C , \qquad ( 3 )$$

where kᵣ and k_{C} are the first and the last sample of the FFR pullback curve, respectively, C is the number of change points, and B is the fixed penalty term (set equal to 0.1 in this study). The minimization of the cost function was obtained implementing an algorithm based on dynamic programming with early abandonment Killick, R. et al (2012) J. Am. Stat. Assoc. 107, 1590-1598.

It is thus clear that the one or more change points are configured to divide the FFR pullback curve, in two or more segments, in which the change points define the endpoint between two segments, or in other words between two neighboring segments. It is further clear that said segments according to the embodiment shown correspond to a linear function between the bordering change points, or in other words correspond to linearized segments. It is clear the FFR pullback curve corresponds to a set of fractional flow reserve (FFR) values obtained at different positions of a coronary vessel between the ostium and the most distal part of the coronary vessel. It is thus clear that each segment extends between a proximal point of the segment and a distal point of the segment, and corresponds to a subset comprising the FFR values obtained at different positions between said proximal point of the segment and the distal point of the segment, of the set of FFR values obtained at different positions of a coronary vessel between the ostium and the most distal part of the coronary vessel. In other words, each linearized segment corresponds to a linearized subset of the FFR pullback curve, or in other words the corresponding set of FFR values, bordered by at least one change point. It is clear that the proximal point of the segment corresponds to a position closer to the ostium of the vessel of the set of FFR values. It is clear that the distal point of the segment corresponds to a position closer to the distal part of the vessel of the set of FFR values. It is further clear that the two or more segments, according to the embodiment shown, extend between the ostium and the most distal part of the coronary vessel in such a way that a first segment extends between the ostium and a first change point, and a last segment extends between the last change point and the most distal part of the coronary vessel. It is clear that in an embodiment in which there are two or more change points detected in the FFR pullback curve, there will be a corresponding sequence of one or more segments which extends between this first segment and the last segment.

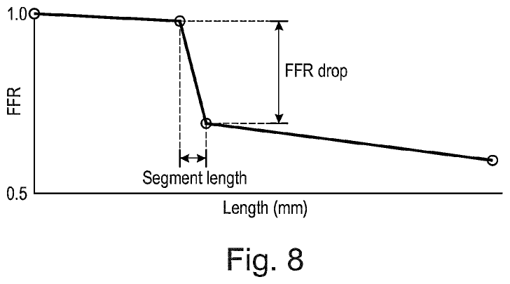

Once the piece-wise linearization of FFR pullback curve has been carried out, each linearized segment of the curve was then characterized by two quantities (FIG. 8): (1) the FFR drop, defined as the difference between the FFR values at the distal and at the proximal point of the segment; (2) the segment length, defined as the distance along the vessel axis between the distal point of the segment and the proximal point of the segment.

On each segment a third quantity, the segment slope, was defined as the ratio between FFR drop and segment length.

### 5.2 Automatic Segments Classification

Two independent observers (CaC, SN) adjudicated by visual inspection each segment of the piece-wise linearized of FFR pullback curve as belonging to one of five segment phenotypes: (1) healthy segment; (2) focal disease segment; (3) diffuse disease segment; (4) pressure recovery segment; (5) artifact.

The automatic adjudication of each segment of the piece-wise linearized of FFR pullback curves to one of the defined five segment phenotypes was based on the application of a logistic regression model on FFR pullback curves segments from the derivation cohort (81 Patients). Technically, the logistic regression model was built on segments that obtained the same adjudication from the two observers (216 segments), while segments manually classified as artefacts were not considered. Two independent variables were considered in the logistic regression model to discriminate the segments: FFR drop and segment length. It is clear that in this way suitable first and second classification threshold functions were derived for the logistic regression model to discriminate healthy segments from diseased segments, and focal diseased segments from diffuse diseased segments as mentioned above. It is clear that the first and second classification threshold functions are different. It is further clear that the first classification threshold function, according to the embodiment shown, operates on all the segments. It is further clear that the second classification threshold function, according to the embodiment shown, only operates on the segments that are classified as diseased by means of the first classification function. It is clear that any other suitable classification threshold functions might be derived, from any suitable data set, such as a suitable derivation cohort, comprising any suitable number of patients, from which any suitable number of observers, provides a suitable classification of the segments. According to an embodiment, the classification threshold functions, such as for example for classifying the segments by means of the logistic regression model, can be derived from any suitable supervised, data-driven approach. It is clear that according to alternative embodiments any suitable training dataset, comprising any suitable size could be used to determine such suitable classification threshold functions for a suitable model for automatically classifying the segments, for example based on FFR drop, segment length, slope or any other suitable parameter of the segments.

Since the logistic regression provides a binary separation, a two-steps approach was implemented for the piece-wise linearized FFR pullback curve segments automatic adjudication:
step 1: separation between healthy and all (grouping focal and diffuse) diseased segments.
step 2: separation between focal and diffuse diseased segments.

In the healthy segment phenotype, pressure recovery segments were identified as the ones meeting all the following criteria: they have a positive FFR drop, they are contiguous to a diseased segment, and they are shorter than 20 mm.

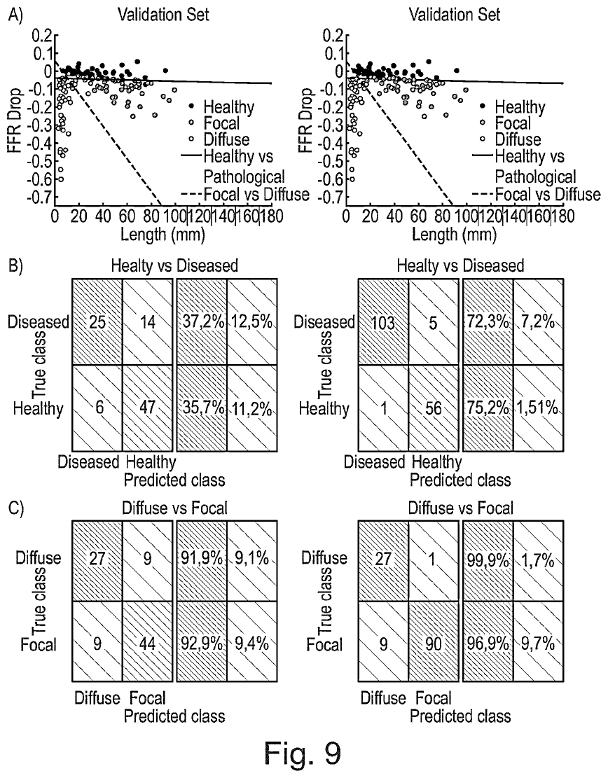

The performance of the automatic adjudication was evaluated by comparison with the manual adjudication by the two observers (CaC; SN) on piece-wise linearized FFR pullback curves belonging to the validation cohort (50 patients, 179 segments). The results of the automatic adjudication are reported in FIG. 9 in terms of accuracy, sensitivity, specificity.

### 5.3 Automatic Classifier Performances

The ability of the proposed automatic adjudication method in discriminating healthy, focal, and diffuse disease segments clearly emerges (FIG. 9, panel A). Concerning the adjudication of healthy vs. pathological segments (FIG. 9, panel B), the automatic adjudication provided an excellent performance in terms of accuracy (87.7% for CaC, 94.6% for SN) sensitivity (87.2% for CaC, 92.8% for SN) and specificity (88.7% for CaC, 98.2% for SN respectively). Concerning the adjudication of focal vs. diffuse disease (FIG. 9, panel C), the automatic adjudication performance was also outstanding in terms of accuracy (94.6% for CaC, 96.4% for SN), sensitivity (91.9% for CaC, 98.3% for SN) and specificity (93.6% for CaC, 94.3% for SN). It is clear that pathological segments are diseased segments.

### 6. FAM Sensitivity to Serial Lesions

Sensitivity of FAM_{QCA} and FAM_{OCT} to serial lesions was tested with two approaches: (1) considering only the contiguous segments in the definition of the functional length and (2) excluding serial lesions from the analysis. When considering only the contiguous segments to define the functional length, both FAM_{QCA} and FAM_{OCT} were still correlated with FFR relative gain after PCI (r=0.606, 95% CI 0.387 to 0.760, p<0.001, FIG. 10A and r=0.560, 95% CI 0.326 to 0.729, p<0.001, FIG. 10C). Excluding serial lesions (10 cases excluded for QCA and 8 cases excluded for OCT), both FAM_{QCA} and FAM_{OCT} were still correlated with FFR relative gain after PCI (r=0.708, 95% CI 0.502 to 0.838, p<0.001, FIG. 10B and r=0.679, 95% CI 0.400 to 0.8427, p<0.001, FIG. 10D).

### 7. Functional-Anatomical Mismatch (FAM)

The automatic piece-wise linearization and classification of the FFR curve segments allowed to derive the extent of the functional disease, namely the functional length, which was expressed in millimeters. In detail, the functional length of disease for each coronary artery was obtained as the summation of the length of all linearized FFR curve segments classified as diseased by the algorithm. In the presence of serial or multiple lesions (i.e. functionally diseased segments separated by functionally healthy segments), the functional length was considered as the sum of all (i.e., contiguous and non-contiguous) diseased segments.

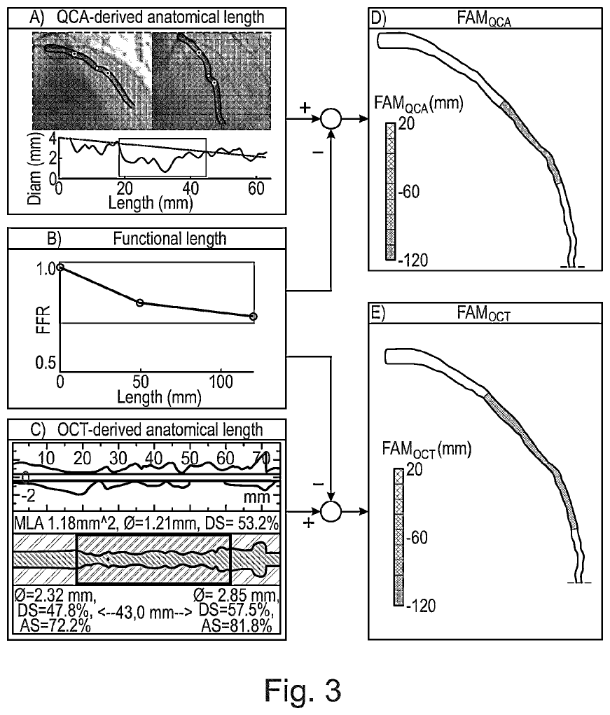

The difference between the anatomical and the functional length of CAD was defined as Functional Anatomical Mismatch (FAM) (FIG. 1D). This quantity allows identifying two lesion endotypes: (1) functional disease circumscribed within the anatomical defined lesion (i.e. FAM>0), and (2) functional disease extending beyond the anatomical defined lesion (FAM<0). A negative FAM translates pressure losses outside the anatomical lesion. Therefore, a positive FAM represents focal CAD where the functional length of disease is restricted to the lesion length, whereas a negative FAM value points to the presence of functional disease outside the anatomical lesion (FIG. 2). For visualization purposes, FAM values were colored-coded using the 3D QCA geometries inside the anatomical lesion with positive values shown in red and negative values in blue (FIG. 2). As the anatomical length of CAD can be derived from QCA or OCT, two FAM values were calculated, namely FAM_{QCA} and FAM_{OCT} (FIG. 3). In addition, the proportion of pressure loss contained within the anatomical lesion defined the FFR drop attributable to the QCA or OCT-derived anatomical lesion relative to the FFR drop of the entire vessel (i.e. FFR drop within QCA or OCT lesion, respectively).

PCI was performed following standard of care guided by FFR and OCT, both executed before and after stent implantation. Intraprocedural PCI guidance or stent optimizations based on either physiology or imaging were left at operator's discretion. New generation DES were used in all cases. To quantify the impact of PCI, the relative functional gain was defined as post PCI FFR minus pre-PCI FFR divided by 1−pre-PCI FFR.

### 8. Statistical Analysis

Continuous data are presented as mean (±SD) or median [25th-75th percentiles]. Categorical data are presented as counts and proportions (%). Differences were evaluated using the univariate Mann-Whitney non-parametric U test. Spearman's correlation coefficients were calculated to assess the relationship between FAM and post-PCI FFR. Agreement between observers was assessed by the intraclass correlation coefficient (ICC). The optimal cutoff values of FAM to predict relative functional gain were calculated using receiver operating characteristic (ROC) curves. The discriminant ability of FAM value to predict optimal post-PCI physiologic results was evaluated with area under curve (AUC). Optimal relative functional gain was defined as an increase in epicardial conductance greater than 50%.

1. A computer device for quantifying the extent of functional coronary artery disease (CAD) comprising a processor configured to:
i) process a set of fractional flow reserve (FFR) values obtained at different positions of a coronary vessel between the ostium and the most distal part of the coronary vessel, and
ii) classify the coronary vessel in healthy segments, focal diseased segments and/or diffused diseased segments by carrying out a piece-wise linearization of said FFR data by applying an automated change-points detection algorithm.

2. The computer device according to claim 1, wherein the computer device further comprises a display configured to display said healthy segments, focal diseased segments and/or diffused diseased segments, optionally on an image of the coronary artery, optionally wherein the displayed image of the coronary artery is a 2-dimensional image.

3. The computer device according to claim 1, wherein the automated change-point detection algorithm is configured to detect one or more change points in the set of FFR values, such that said change points each correspond to a position along the coronary vessel where an attribute of the set of FFR values changes, wherein:
said one or more change points are configured to divide the set of FFR values in two or more segments, in which each change point defines an endpoint between two segments; and
said two or more segments, each corresponding to a linearized subset of the set of FFR values obtained at different positions along the coronary vessel between a proximal point of the segment and a distal point of the segment.

4. The computer device according to claim 3, wherein:
said attribute is an average value and/or a slope; and/or
said two or more segments are characterized by the following quantities:
FFR drop, which is the difference between the FFR value at the distal point and the FFR value at the proximal point of the segment; and
segment length, which is as the distance along the coronary vessel axis between the distal point of the segment and the proximal point of the segment; and
optionally segment slope, which is the ratio between the FFR drop and the segment length.

5. The computer device according to claim 1, wherein the computer device is further configured to classify the coronary vessel such that:
segments are classified as healthy segments or as diseased segments by means of a predetermined first classification threshold function based on the FFR drop, the segment length and/or the segment slope of the segments; and
optionally, diseased segments are classified as:
focal diseased segments or as diffuse diseased segments by means of a predetermined second classification threshold function based on the FFR drop, segment length and/or segment slope of the segments; and
optionally, segments as classified as healthy when said segments exhibit a positive FFR drop and when said segments are contiguous to a diseased segment and said segments are shorter than 30 mm; and
optionally the computer device further comprises a logistic regression model configured to automatically discriminate each segment as a healthy segment, a focal diseased segment and/or a diffuse diseased segment, optionally a two-variables logistic regression based on the FFR drop, the segment length and/or the slope of the segment, optionally, wherein the logistic regression model is determined from visual adjudication of a derivation cohort, configured to discriminate between healthy and diseased segments, and further to discriminate between focal diseased segments and diffuse diseased segments.

6. The computer device according to claim 1, wherein said automated change-points detection algorithm is configured to operate based on a penalized parametric global method.

7. The computer device according to claim 2, wherein the display is further configured to display the image of the coronary artery in a 2-dimensional image.

8. The computer device according to claim 1, further configured to obtain the set of FFR values from:
a pull-back curve; or
a 3-dimensional quantitative coronary angiography; or
a CT scan; or
intravascular imaging, optionally an optical coherence tomography (OCT) or an intravascular ultrasound (IVUS); or
the combination between coronary angiography and intravascular imaging; or
the combination of a CT scan and intravascular imaging; or
computational fluid dynamics simulations applied to a 3D model of the coronary vessel as reconstructed from medical imaging, optionally wherein the medical imaging comprises: a 3-dimensional quantitative coronary angiography, a CT scan, an OCT or an IVUS.

9. The computer device according to claim 1, wherein the computer device is further configured to predict the response to a percutaneous coronary intervention (PCI) by said quantifying of the extent of functional CAD, and/or wherein the computer device is further configured to quantify the extent of functional CAD as the sum of the lengths of the diseased segments.

10. The computer device according to claim 1, wherein the computer device is further configured to select a mammal suffering from coronary artery disease (CAD) to be eligible for a percutaneous coronary intervention (PCI) by said quantifying of the extent of functional CAD, and selecting a mammal when the extent of functional disease in the coronary artery is smaller than the extent of anatomical disease in the coronary artery; and/or
wherein the computer device is further configured to calculate a Functional Anatomical Mismatch (FAM) as the difference between the extent of anatomical CAD and the extent of functional, thereby identifying two lesion endotypes: (1) functional CAD circumscribed within the anatomical CAD when FAM>0, and (2) functional CAD extending beyond the anatomical CAD when FAM<0.

11. The computer device according to claim 1, wherein the computer device is configured to operate offline.

12. The computer device according to claim 1, wherein the computer device is configured to perform said automatic classification.

13. A computer-implemented method to quantify the extent of functional coronary artery disease (CAD) comprising the following steps:
i) processing a set of fractional flow reserve (FFR) values obtained at different positions of a coronary vessel between the ostium and the most distal part of the coronary vessel,
ii) classifying the coronary vessel in healthy segments, focal diseased segments and/or diffused diseased segments by carrying out a piece-wise linearization of said FFR data by applying an automated change-points detection algorithm, and optionally
iii) displaying said healthy, focal and/or diffused diseased fragments on an image of the coronary artery, and optionally said automated change-points detection algorithm is based on a penalized parametric global method.

14. The computer-implemented method according to claim 13, wherein said method further comprises the step of obtaining the set of FFR values from a pull-back curve, or 3-dimensional quantitative coronary angiography, or a CT scan, or intravascular imaging, or the combination between coronary angiography and intravascular imaging or the combination of a CT scan and intravascular imaging.

15. A computer-implemented method for developing an automated classifier for use in the computer device according to claim 1 for performing the classification of the coronary vessel in healthy focal and/or diffused diseased segments, wherein:
the automatic classifier is developed based on logistic regression; and
optionally the logistic regression is determined from visual adjudication of a derivation cohort, configured to discriminate between healthy and diseased segments, and further to discriminate between focal diseased segments and diffuse diseased segments.

16. The computer device according to claim 5, wherein said segments that are shorter than 30 mm are shorter than 25 mm.

17. The computer device according to claim 5, wherein said segments that are shorter than 30 mm are shorter than 20 mm.

18. The computer-implemented method according to claim 15, wherein the logistic regression is two-variables logistic regression based on the FFR drop, the segment length and/or the slope of the associated segment.

19. A computer-implemented method for developing an automated classifier for use in the computer-implemented method according to claim 13 for performing the classification of the coronary vessel in healthy focal and/or diffused diseased segments, wherein:
the automatic classifier is developed based on logistic regression; and
optionally the logistic regression is determined from visual adjudication of a derivation cohort, configured to discriminate between healthy and diseased segments, and further to discriminate between focal diseased segments and diffuse diseased segments.

20. The computer-implemented method according to claim 19, wherein the logistic regression is two-variables logistic regression based on the FFR drop, the segment length and/or the slope of the associated segment.
